(19) United States
(12) Patent Application Publication
(10) Pub. No.: US 2012/0245117 A1
(43) Pub. Date: Sep. 27, 2012

(54) METHODS OF TREATING SQUAMOUS CELL LUNG CANCER WITH 4-IODO-3-NITROBENZAMIDE IN COMBINATION WITH GEMCITABINE AND CARBOPLATIN

(76) Inventor: Charles BRADLEY, Half Moon Bay, CA (US)

(21) Appl. No.: 13/411,268

(22) Filed: Mar. 2, 2012

Related U.S. Application Data
(60) Provisional application No. 61/449,592, filed on Mar. 4, 2011.

(51) Int. Cl. A61K 31/7068; A61P 35/04; A61P 35/00

(52) U.S. Cl. 514/49

(57) ABSTRACT

In one aspect, the present invention provides a method of treating squamous cell lung cancer in an individual comprising administering to the individual having lung cancer an effective amount of 4-iodo-3-nitrobenzamide or a metabolite thereof or pharmaceutically acceptable salt thereof; gemcitabine; and carboplatin.

### CROSS-REFERENCE TO RELATED APPLICATIONS

This application claims the priority benefit of U.S. provisional application Ser. No. 61/449,592, filed Mar. 4, 2011, which is incorporated herein by reference in its entirety.

### TECHNICAL FIELD

The present disclosure relates to methods of treating squamous cell lung cancer by using a) 4-iodo-3-nitrobenzamide, a metabolite thereof, or a pharmaceutically acceptable salt thereof, b) gemcitabine, and (c) carboplatin.

### BACKGROUND OF THE INVENTION

Cancer is a major public health problem in the United States and many other parts of the world, with more than 1.4 million cases of cancer reported each year in the U.S. causing one in four deaths in the U.S. annually. See, e.g., Jemal et al., “Cancer Statistics, 2009,” CA Cancer J. Clin. 59:225-249 (2009). Cancer is a complex family of diseases affecting nearly every tissue in the body and characterized by aberrant control of cell growth. While a number of first line therapies for the treatment of different types of cancer have been deployed with varying degrees of success, including surgical resection, radiation therapy, chemotherapy, and hormone therapy, it remains the second leading cause of death in the U.S., with an estimated 562,000 Americans dying from cancer every year.

Lung cancer is one of the leading causes of cancer-related mortality in the U.S. Lung cancer can be visualized on chest x-ray and by computed tomography (CT) scan; radiologic diagnosis is generally confirmed via biopsy. Treatment and prognosis depend upon the histological type of cancer, the stage (degree of spread), and the patient's performance status. Possible treatments include surgery, chemotherapy, and radiotherapy, but even with treatment, the five-year survival rate is relatively low at ˜14%.

The current standard of care for lung cancer consists of so-called “platinum doublets.” A platinum doublet is a combination therapy comprising a platinum compound (e.g., cisplatin, carboplatin or oxaliplatin) and a second chemotherapeutic agent (e.g., paclitaxel, docetaxel, or gemcitabine and the like). Combination therapies based on antimetabolites (e.g., citabine, capecitabine, gemcitabine or valopicitabine) have also been used, though platinum doublets remain the first line therapy of choice.

Squamous cell lung cancer (SqCLC) has proven more challenging to treat. Unlike other cancer types, anti-angiogenic strategies have failed. In addition, at least one drug formerly used to treat SqCLC—Eli Lilly's Pemetrexed ((S)-2-[4-[2-(4-amino-2-oxo-3,5,7-triazabicyclo[4.3.0]nona-3,8,10-trien-9-yl)ethyl]benzoyl]aminopentanedioic acid)—is no longer approved for use with this patient population. The combination of CP-751,871 (an anti-IGF-1R monoclonal antibody) with carboplatin/paclitaxel showed a strong response rate of ˜78% in SqCLC patients, but also showed strong toxicity, while a combination of cetuximab (a chimeric anti-EGFR monoclonal antibody sold under the trade name Erbitux) with cisplatin/vinorelbine improved median overall survival (10.2 months vs. 8.9 months), but has not so far received FDA approval. Consequently, more effective chemotherapeutic strategies are needed for this large patient population.

All references cited herein, including patent applications and publications, are incorporated by reference in their entirety.

### BRIEF SUMMARY OF THE INVENTION

Provided herein are methods of treating squamous non-small cell lung cancer (“squamous cell lung cancer”) (e.g., stage IV squamous cell lung cancer) in an individual, comprising administering to the individual having squamous cell lung cancer an effective amount of: (i) 4-iodo-3-nitrobenzamide, a metabolite thereof, or a pharmaceutically acceptable salt thereof; (ii) gemcitabine; and (iii) carboplatin.

In some embodiments, the squamous cell lung cancer is any of stage 0, I, II, III, or IV. In some embodiments, the squamous cell lung cancer is stage IV. For example, there is provided a method of treating stage IV squamous cell lung cancer in an individual, comprising administering to the individual having squamous cell lung cancer an effective amount of: (i) 4-iodo-3-nitrobenzamide, a metabolite thereof, or a pharmaceutically acceptable salt thereof; (ii) gemcitabine; and (iii) carboplatin.

In some embodiments of any one of the methods provided herein, the squamous cell lung cancer is metastatic. In some embodiments, the squamous cell lung cancer is locally advanced. In some embodiments, the individual has distant metastases. In some embodiments, the individual has disseminated metastases. In some embodiments, the individual has malignant pleural or pericardial effusion. In some embodiments, the squamous cell lung cancer is recurrent. In some embodiments, the individual has squamous cell bronchogenic carcinoma.

In some embodiments of any one of the methods provided herein, the individual has received prior chemotherapy (e.g., prior chemotherapy for lung cancer such as squamous cell lung cancer). In some embodiments, the individual has not received prior chemotherapy (e.g., prior chemotherapy for lung cancer such as squamous cell lung cancer). In some embodiments, the individual has not received prior chemotherapy comprising 4-iodo-3-nitrobenzamide, a metabolite thereof, or a pharmaceutically acceptable salt thereof. In some embodiments, the individual has not received prior chemotherapy comprising gemcitabine. In some embodiments, the individual has not received prior chemotherapy comprising carboplatin. In some embodiments, the individual has received prior chemotherapy comprising carboplatin in the adjuvant setting. In some embodiments, the individual has received carboplatin in the adjuvant setting for lung cancer. In some embodiments, the individual has received carboplatin in the adjuvant setting for early stage lung cancer.

In some embodiments of a method described herein, the individual has newly diagnosed stage IV squamous cell lung cancer. In some embodiments, the individual has stage IV squamous cell lung cancer at diagnosis or recurrent after resection of the squamous cell lung cancer. In some embodiments, the individual has previously received adjuvant chemotherapy for early stage lung cancer. In some embodiments, the individual has not previously received adjuvant chemotherapy for early stage lung cancer. In some embodiments, the individual has not previously received systemic treatment for advanced squamous cell lung cancer. In some embodiments, the individual has previously received adjuvant therapy for early stage lung cancer and the adjuvant therapy ended at least 12 months ago. In some embodiments, the individual has not previously received a therapy comprising chemotherapy and radiation therapy for locally advanced squamous cell lung cancer.

In some embodiments of any one of the methods described herein, 4-iodo-3-nitrobenzamide or a metabolite thereof or a pharmaceutically acceptable salt thereof is administered at any of about 0.1 to about 50 mg/kg, about 1 to about 50 mg/kg, about 2 to about 40 mg/kg, about 3 to about 30 mg/kg, about 4 to about 20 mg/kg, or about 4 to about 15 mg/kg (e.g., about 5.6 mg/kg). In some embodiments, gemcitabine is administered at any of about 100 to about 5000 mg/m², about 200 to about 4000 mg/m², about 300 to about 3000 mg/m², about 400 to about 2000 mg/m², about 500 to about 1500 mg/m², about 800 to about 1500 mg/m², or about 800 to about 1200 mg/m² (e.g., about 1000 mg/m²). In some embodiment, carboplatin is administered at any of about AUC 1 mg/ml·minute (“AUC 1”) to about AUC 8, about AUC 2 to about AUC 6, about AUC 3 to about AUC 6, or about AUC 4 to about AUC 6 (e.g., about AUC5).

In some embodiments of any one of the methods described herein, the method comprises at least one treatment cycle comprising (i) 4-iodo-3-nitrobenzamide or a metabolite thereof or a pharmaceutically acceptable salt thereof, (ii) gemcitabine, and (iii) carboplatin. For example, the method may comprise at least 2 (at least about any of 3, 4, 5, 6, 7, 8, 9, or 10) treatment cycles. A treatment cycle may be a period of about any of 1 week, 10 days, 2 weeks, 3 weeks, 4 weeks, 5 weeks, 6 weeks, 7 weeks, 8 weeks, 9 weeks, or 10 weeks.

In some embodiments of any one of the methods provided herein, the effective amount is administered over a 21-day treatment cycle, wherein (i) the effective amount of carboplatin is administered to the individual at about 5 mg/ml·minute (AUC 5) on day 1 of the treatment cycle; (ii) the effective amount of gemcitabine is administered to the individual at a dose of about 1000 mg/m² on days 1 and 8 of the treatment cycle; and (iii) the effective amount of 4-iodo-3-nitrobenzamide or a metabolite thereof or a pharmaceutically acceptable salt thereof is administered to the individual at a dose of about 5.6 mg/kg twice weekly on days 1, 4, 8, and 11 of the treatment cycle. In some embodiments, there is provided a method of treating squamous cell lung cancer (e.g., stage IV squamous cell lung cancer) in an individual, comprising administering to the individual having squamous cell lung cancer an effective amount of: (i) 4-iodo-3-nitrobenzamide, a metabolite thereof, or a pharmaceutically acceptable salt thereof; (ii) gemcitabine; and (iii) carboplatin, wherein the effective amount is administered over a 21-day treatment cycle, wherein (i) carboplatin is administered to the individual at about 5 mg/ml·minute (AUC 5) on day 1 of the treatment cycle; (ii) gemcitabine is administered to the individual at about 1000 mg/m² on days 1 and 8 of the treatment cycle; and (iii) 4-iodo-3-nitrobenzamide or a metabolite thereof or a pharmaceutically acceptable salt thereof is administered to the individual at about 5.6 mg/kg on days 1, 4, 8, and 11 of the treatment cycle. In some embodiments, the individual receives at least 2 treatment cycles (e.g., about any of or at least about any of 2, 3, 4, 5, 6, 7, 8, 9, or 10 cycles).

In some embodiments, 4-iodo-3-nitrobenzamide or a metabolite thereof or a pharmaceutically acceptable salt thereof is administered intravenously to the individual. In some embodiments, gemcitabine is administered intravenously to the individual. In some embodiments, carboplatin is administered intravenously to the individual.

In some embodiments, the effective amount produces at least one therapeutic effect selected from the group consisting of reduction in size of a lung tumor, reduction in metastasis, complete remission, partial remission, stable disease, increase in overall response rate, or a pathologic complete response. In some embodiments, a comparable clinical benefit rate (CBR=CR (complete remission)+PR (partial remission)+SD (stable disease)≧6 months) is obtained compared to treatment with said gemcitabine and said carboplatin administered without 4-iodo-3-nitrobenzamide. In some embodiments, the improvement of clinical benefit rate is about 20% or higher.

In some embodiments, the method further comprises surgery, radiation therapy, chemotherapy, gene therapy, DNA therapy, viral therapy, RNA therapy, adjuvant therapy, neoadjuvant therapy, immunotherapy, nanotherapy or a combination thereof. In some embodiments, the method further comprises administering to the individual gamma irradiation.

In some embodiments, the individual having squamous cell lung cancer has a history of smoking. In some embodiments, the individual having squamous cell lung cancer does not have a history of smoking.

Also provided herein are methods of marketing or promoting one or more of (i) 4-iodo-3-nitrobenzamide, a metabolite thereof, or a pharmaceutically acceptable salt thereof; (ii) gemcitabine; and (iii) carboplatin for treating an individual having squamous cell lung cancer. For example, provided herein is a method for marketing or promoting 4-iodo-3-nitrobenzamide, a metabolite thereof, or a pharmaceutically acceptable salt thereof for use in treating squamous cell lung cancer, the method comprising informing a target audience about the use of 4-iodo-3-nitrobenzamide, a metabolite thereof, or a pharmaceutically acceptable salt thereof for treating an individual having squamous cell lung cancer, wherein the treatment comprises administering an effective amount of (i) 4-iodo-3-nitrobenzamide, a metabolite thereof, or a pharmaceutically acceptable salt thereof; (ii) gemcitabine; and (iii) carboplatin. Also provided herein is a method for marketing or promoting (i) 4-iodo-3-nitrobenzamide, a metabolite thereof, or a pharmaceutically acceptable salt thereof; (ii) gemcitabine; and/or (iii) carboplatin for use in treating squamous cell lung cancer, the method comprising informing a target audience about the use of (i) 4-iodo-3-nitrobenzamide, a metabolite thereof, or a pharmaceutically acceptable salt thereof; (ii) gemcitabine; and (iii) carboplatin for treating an individual having squamous cell lung cancer, wherein the treatment comprises administering an effective amount of (i) 4-iodo-3-nitrobenzamide, a metabolite thereof, or a pharmaceutically acceptable salt thereof; (ii) gemcitabine; and (iii) carboplatin. In some embodiments of any one of the methods described herein, the method further comprises providing a kit comprising any one or more of (i) 4-iodo-3-nitrobenzamide, a metabolite thereof, or a pharmaceutically acceptable salt thereof; (ii) gemcitabine; and (iii) carboplatin to the target audience. In some embodiments, the kit comprises 4-iodo-3-nitrobenzamide, a metabolite thereof, or a pharmaceutically acceptable salt thereof. In some embodiments, the kit comprises (i) 4-iodo-3-nitrobenzamide, a metabolite thereof, or a pharmaceutically acceptable salt thereof; (ii) gemcitabine; and (iii) carboplatin. In some embodiments of any one of the methods described herein, the method further comprises providing instructions to the target audience regarding administration of an effective amount of (i) 4-iodo-3-nitrobenzamide, a metabolite thereof, or a pharmaceutically acceptable salt thereof; (ii) gemcitabine; and (iii) carboplatin to the individual. In some embodiments, the information provided to the target audience indicates that the treatment is effective in treating squamous cell lung cancer (such as stage IV squamous cell lung cancer). In some embodiments, the methods of marketing or promoting is followed by the treatment of the individual by administering an effective amount of (i) 4-iodo-3-nitrobenzamide, a metabolite thereof, or a pharmaceutically acceptable salt thereof; (ii) gemcitabine; and (iii) carboplatin to the individual. In some embodiments, the target audience is a health professional (e.g., a physician, a nurse, etc.), an individual having lung cancer (such as squamous cell lung cancer), or a person involved with the individual's care/treatment (e.g., relative, care-giver, etc.). In some embodiments, informing a target audience may be conducted by any means available, such as providing written materials (e.g., in either paper or digital form) and/or oral presentation(s) (e.g., either in person (live), as recorded, on television or internet). In some embodiments, the written material is in a package insert accompanying 4-iodo-3-nitrobenzamide, a metabolite thereof, or a pharmaceutically acceptable salt thereof in a kit.

Also provided herein are kits comprising 4-iodo-3-nitrobenzamide, a metabolite thereof, or a pharmaceutically acceptable salt thereof, and instructions for using 4-iodo-3-nitrobenzamide, a metabolite thereof, or a pharmaceutically acceptable salt thereof in combination with gemcitabine and carboplatin for treatment of squamous cell lung cancer in an individual. The instruction may be according to any one of the methods provided herein. In some embodiments, the instructions comprise administering an effective amount of (i) 4-iodo-3-nitrobenzamide, a metabolite thereof, or a pharmaceutically acceptable salt thereof, (ii) gemcitabine, and (iii) carboplatin over a 21-day treatment cycle to the individual, wherein (i) carboplatin is administered to the individual at about 5 mg/ml·minute (AUC 5) on day 1 of the treatment cycle; (ii) gemcitabine is administered to the individual at about 1000 mg/m² on days 1 and 8 of the treatment cycle; and (iii) 4-iodo-3-nitrobenzamide or a metabolite thereof or a pharmaceutically acceptable salt thereof is administered to the individual at about 5.6 mg/kg on days 1, 4, 8, and 11 of the treatment cycle. In some embodiments, the squamous cell lung cancer is stage IV. In some embodiments, the instructions are included in a package insert or a label in the kit.

Also provided herein are kits comprising (i) 4-iodo-3-nitrobenzamide, a metabolite thereof, or a pharmaceutically acceptable salt thereof, (ii) gemcitabine, and/or (iii) carboplatin. In some embodiments, the kit further comprises instructions for using (i) 4-iodo-3-nitrobenzamide, a metabolite thereof, or a pharmaceutically acceptable salt thereof; (ii) gemcitabine; and (iii) carboplatin for treatment of squamous cell lung cancer in an individual. In some embodiments, there is provided a kit comprising (i) 4-iodo-3-nitrobenzamide, a metabolite thereof, or a pharmaceutically acceptable salt thereof; (ii) gemcitabine; and (iii) carboplatin, and an instruction for using (i) 4-iodo-3-nitrobenzamide, a metabolite thereof, or a pharmaceutically acceptable salt thereof; (ii) gemcitabine; and (iii) carboplatin in the treatment of squamous lung cancer in an individual. The instruction may be according to any one of the methods provided herein. In some embodiments, the instructions comprise administering an effective amount of (i) 4-iodo-3-nitrobenzamide, a metabolite thereof, or a pharmaceutically acceptable salt thereof, (ii) gemcitabine, and (iii) carboplatin over a 21-day treatment cycle to the individual, wherein (i) carboplatin is administered to the individual at about 5 mg/ml·minute (AUC 5) on day 1 of the treatment cycle; (ii) gemcitabine is administered to the individual at about 1000 mg/m² on days 1 and 8 of the treatment cycle; and (iii) 4-iodo-3-nitrobenzamide or a metabolite thereof or a pharmaceutically acceptable salt thereof is administered to the individual at about 5.6 mg/kg on days 1, 4, 8, and 11 of the treatment cycle. In some embodiments, the squamous cell lung cancer is stage IV. In some embodiments, the instructions are included in a package insert or a label in the kit.

Also provided are articles of manufacture comprising the compositions described herein in suitable packaging. Suitable packaging for compositions described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed.

Also provided herein are uses of any one of the pharmaceutical compositions described herein for the manufacture of a medicament for treating squamous cell lung cancer (e.g., stage IV squamous cell lung cancer). In some embodiments, the use is in accordance with any one of the methods described herein.

Also provided herein are uses of 4-iodo-3-nitrobenzamide, a metabolite thereof, or a pharmaceutically acceptable salt thereof for the manufacture of a medicament for treating squamous cell lung cancer in an individual, wherein 4-iodo-3-nitrobenzamide, a metabolite thereof, or a pharmaceutically acceptable salt thereof is administered in combination with gemcitabine and carboplatin to the individual.

Also provided herein are compositions comprising a) 4-iodo-3-nitrobenzamide, or a metabolite thereof, or a pharmaceutically acceptable salt or solvate thereof, b) gemcitabine, and/or c) carboplatin. In some embodiments, there is provided a synergistic composition used for treating squamous cell lung cancer in an individual comprising a) 4-iodo-3-nitrobenzamide, or a metabolite thereof, or a pharmaceutically acceptable salt or solvate thereof, b) gemcitabine, and c) carboplatin.

It is to be understood that one, some, or all of the properties of the various embodiments described herein may be combined to form other embodiments of the present invention. These and other aspects of the invention will become apparent to one of skill in the art.

### DETAILED DESCRIPTION OF THE INVENTION

Provided herein are methods of treating squamous non-small cell lung cancer (“squamous cell lung cancer”) (e.g., stage IV squamous cell lung cancer) in an individual, comprising administering to the individual having squamous cell lung cancer an effective amount of: (i) 4-iodo-3-nitrobenzamide, a metabolite thereof, or a pharmaceutically acceptable salt thereof; (ii) gemcitabine; and (iii) carboplatin. In some embodiments, the squamous cell lung cancer is stage IV.

The term “individual” refers to a mammal and includes, but is not limited to, human, bovine, horse, feline, canine, rodent, or primate. Preferably, the individual is a human. An individual may be a human patient or subject as described herein including the Examples.

As used herein “surgery” refers to any therapeutic or diagnostic procedure that involves methodical action of the hand or of the hand with an instrument, on the body of a human or other mammal, to produce a curative, remedial, or diagnostic effect.

“Radiation therapy” refers to exposing an individual to high-energy radiation, including without limitation x-rays, gamma rays, and neutrons. This type of therapy includes without limitation external-beam therapy, internal radiation therapy, implant radiation, brachytherapy, systemic radiation therapy, and radiotherapy.

“Chemotherapy” refers to the administration of one or more anti-cancer drugs such as, antineoplastic chemotherapeutic agents, chemopreventative agents, and/or other agents to an individual (e.g., an individual with squamous cell lung cancer) by various methods, including intravenous, oral, intramuscular, intraperitoneal, intravesical, subcutaneous, transdermal, buccal, or inhalation or in the form of a suppository. Chemotherapy may be given prior to surgery to shrink a large tumor prior to a surgical procedure to remove it, prior to radiation therapy, or after surgery and/or radiation therapy to prevent the growth of any remaining cancer cells in the body. Chemotherapy may also occur during the course of radiation therapy.

The terms “effective amount” or “pharmaceutically effective amount” refer to a sufficient amount of an agent to provide the desired biological, therapeutic, and/or prophylactic result. That result can be reduction and/or alleviation of one or more of the signs, symptoms, or causes of a disease, or any other desired alteration of a biological system. For example, an “effective amount” for therapeutic uses is the amount of a) 4-iodo-3-nitrobenzamide or a metabolite thereof or a pharmaceutically acceptable salt or solvate thereof; b) gemcitabine; and c) carboplatin, or a composition comprising a) 4-iodo-3-nitrobenzamide or a metabolite thereof or a pharmaceutically acceptable salt thereof; b) gemcitabine; and c) carboplatin required to provide a clinically significant decrease in the squamous cell lung cancer or slowing of progression of the squamous cell lung cancer.

“Metabolite” refers to a compound produced through any in vitro or in vivo metabolic process which results in a product that is different in structure than that of the starting compound. In other words, the term “metabolite” includes the metabolite compounds of 4-iodo-3-nitrobenzamide. A metabolite can include a varying number or types of substituents that are present at any position relative to a precursor compound. In addition, the terms “metabolite” and “metabolite compound” are used interchangeably herein.

By “pharmaceutically acceptable” is meant a material which is not biologically or otherwise undesirable, i.e., the material may be administered to an individual without causing any undesirable biological effects or interacting in a deleterious manner with any of the components of the composition in which it is contained.

The term “treating” and its grammatical equivalents as used herein include achieving a therapeutic benefit and/or a prophylactic benefit. By therapeutic benefit is meant eradication or amelioration of the underlying disorder being treated. For example, in an individual having squamous cell lung cancer, therapeutic benefit includes eradication or amelioration of the underlying squamous cell lung cancer, e.g., slowing of progression of the lung cancer. Also, a therapeutic benefit is achieved with the eradication or amelioration of one or more of the physiological symptoms associated with the underlying disorder (e.g., squamous cell lung cancer) such that an improvement is observed in the individual, notwithstanding the fact that the individual may still be afflicted with the underlying disorder (e.g., squamous cell lung cancer). For prophylactic benefit, a method of the invention may be performed on, or a composition of the invention administered to an individual at risk of developing lung cancer (e.g., squamous cell lung cancer), or to an individual reporting one or more of the physiological symptoms of lung cancer (e.g., squamous cell lung cancer), even though a diagnosis of lung cancer (e.g., squamous cell lung cancer) may not have been made. In some embodiments, the individual being treated has been diagnosed with squamous cell lung cancer.

Treatment or administration in the “adjuvant setting” refers to a subsequent mode of treatment (for example, a subsequent mode of treatment carried out after the primary/definitive therapy).

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise. It is understood that aspects and variations of the invention described herein include “consisting” and/or “consisting essentially of” aspects and variations.

It is understood that aspects and variations of the methods, uses, compositions, formulations, articles of manufacture, kits, medicaments, or unit dosage forms described herein include “consisting” and/or “consisting essentially of” aspects and variations.

As is apparent to one skilled in the art, an individual assessed, selected for, and/or receiving treatment is an individual in need of such activities.

### 4-iodo-3-nitrobenzamide, metabolites thereof and related compounds thereof

4-iodo-3-nitrobenzamide (a small molecule, also known as iniparib or “BA”) and related compounds thereof are described herein. 4-iodo-3-nitrobenzamide or related compounds thereof, metabolites thereof, pharmaceutically acceptable salts thereof may be used for the treatment of squamous cell lung cancer (e.g., stage IV squamous cell lung cancer). Any of the compounds with structure formula Ia or IIa described herein may be used for the treatment of squamous cell lung cancer (e.g., stage IV squamous cell lung cancer). In some embodiments, an effective amount of a compound with structure formula Ia or IIa in combination with gemcitabine and carboplatin is administered to an individual with squamous cell lung cancer. In some embodiments, the compound with structure formula Ia or IIa is 4-iodo-3-nitrobenzamide or a metabolite thereof or a pharmaceutically acceptable salt thereof.

Provided herein are precursor compounds of Formula (Ia)

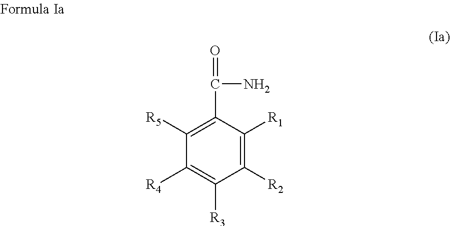

wherein R₁, R₂, R₃, R₄, and R₅ are, independently selected from the group consisting of hydrogen, hydroxy, amino, nitro, nitroso, iodo, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen, at least one of the five substituents is always nitro, and at least one substituent positioned adjacent to a nitro is always iodo, and pharmaceutically acceptable salts, solvates, isomers, tautomers, metabolites, analogs, or pro-drugs thereof. R₁, R₂, R₃, R₄, and R₅ can also be a halide such as chloro, fluoro, or bromo substituents. In some embodiments, at least one of the R₁, R₂, R₃, R₄, and R₅ substituents is always nitro or nitroso and at least one substituent positioned adjacent to the nitro or nitroso is always iodo. In some embodiments, the compound of formula Ia is a compound of formula IA or a metabolite or pharmaceutically acceptable salt, solvate, isomer, or tautomer thereof. In some embodiments, the compound of formula Ia is 4-iodo-3-nitrobenzamide or a metabolite or pharmaceutically acceptable salt, solvate, isomer, or tautomer thereof.

### 4-iodo-3-nitrobenzamide, also known as iniparib or “BA,” has the formula

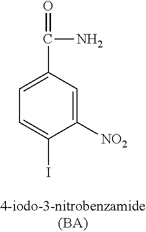

Methods of making 4-iodo-3-nitrobenzamide are known to the field, such as the methods disclosed in U.S. Pat. No. 5,464,871, which is hereby incorporated by reference in its entirety, particularly with respect to the synthetic methods disclosed therein.

Also provided herein are metabolites with the Formula (IIa):

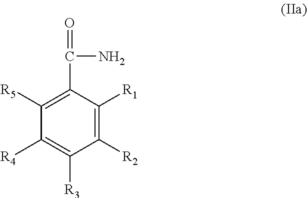

wherein either: (1) at least one of R₁, R₂, R₃, R₄, and R₅ substituent is always a sulfur-containing substituent, and the remaining substituents R₁, R₂, R₃, R₄, and R₅ are independently selected from the group consisting of hydrogen, hydroxy, amino, nitro, iodo, bromo, fluoro, chloro, (C₁-C₆) alkyl, (C₁-C₆) alkoxy, (C₃-C₇) cycloalkyl, and phenyl, wherein at least two of the five R₁, R₂, R₃, R₄, and R₅ substituents are always hydrogen; or (2) at least one of R₁, R₂, R₃, R₄, and R₅ substituents is not a sulfur-containing substituent and at least one of the five substituents R₁, R₂, R₃, R₄, and R₅ is always iodo, and wherein said iodo is always adjacent to a R₁, R₂, R₃, R₄, or R₅ group that is either a nitro, a nitroso, a hydroxyamino, hydroxy or an amino group; and pharmaceutically acceptable salts, solvates, isomers, tautomers, metabolites, analogs, or pro-drugs thereof. In some embodiments, the compounds of (2) are such that the iodo group is always adjacent to a R₁, R₂, R₃, R₄ or R₅ group that is a nitroso, hydroxyamino, hydroxy or amino group. In some embodiments, the compounds of (2) are such that the iodo group is always adjacent to a R₁, R₂, R₃, R₄ or R₅ group that is a nitroso, hydroxyamino, or amino group.

Provided herein are metabolite compounds, each represented by a chemical formula:

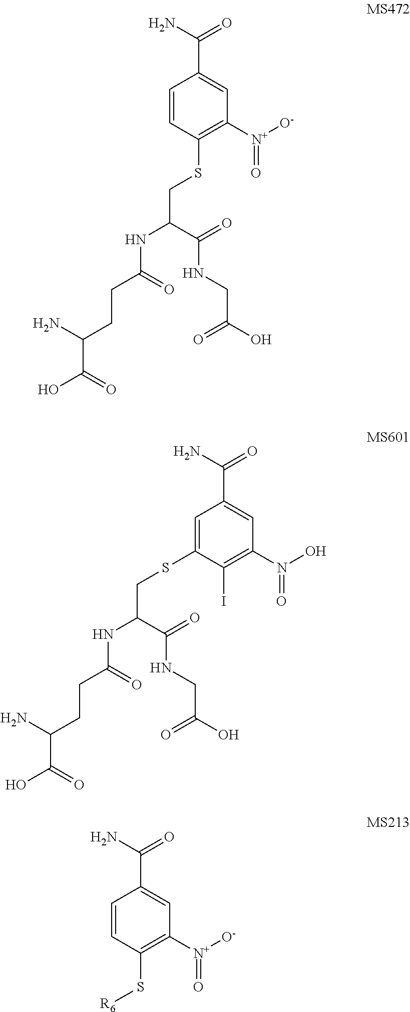

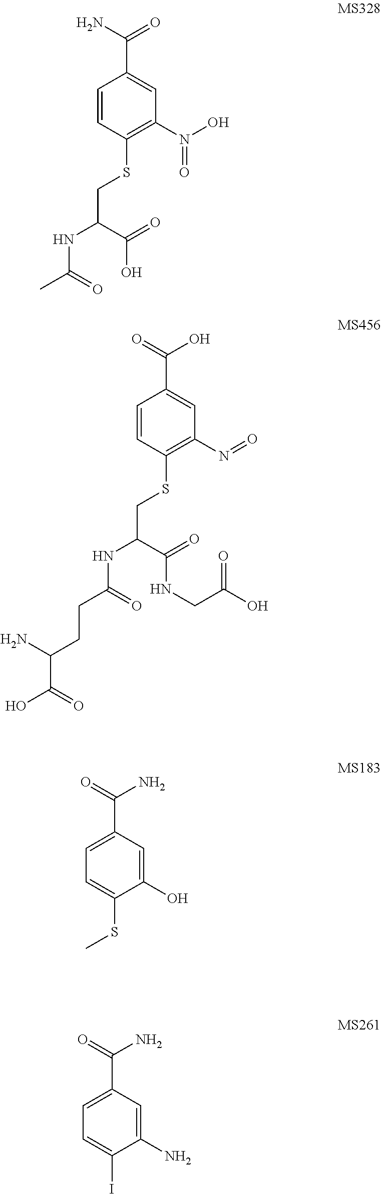

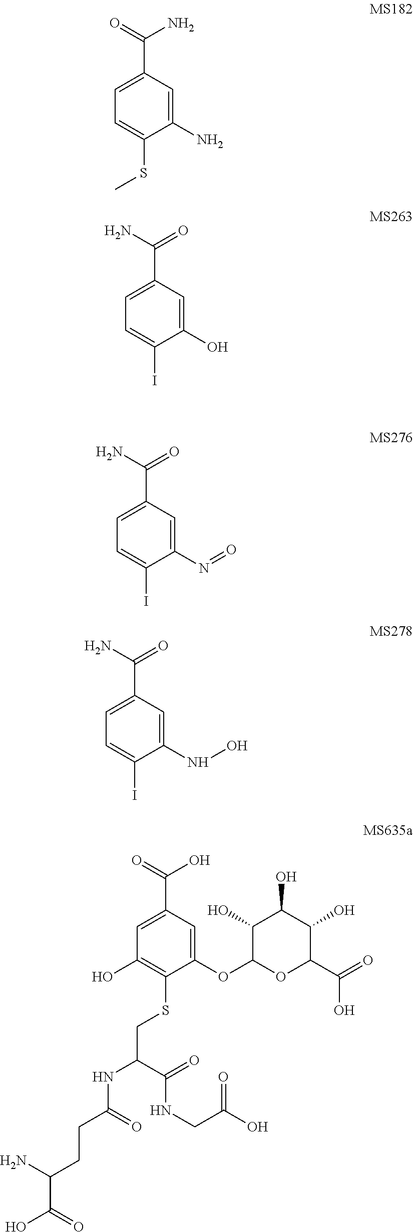

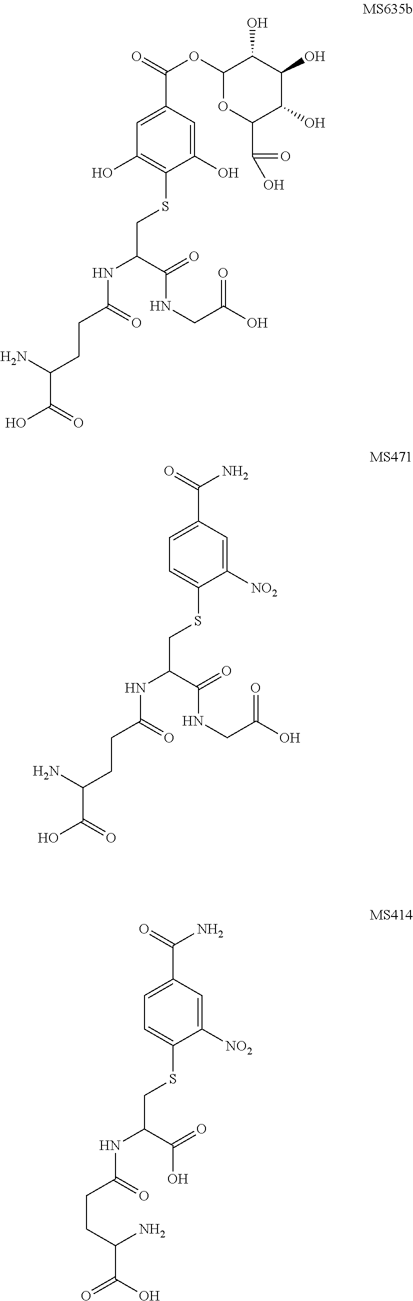

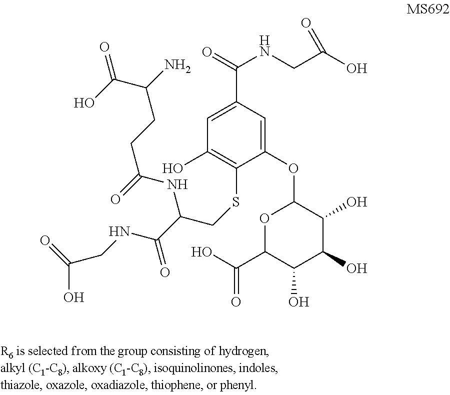

While not being limited to any one particular mechanism, the following provides an example for MS292 metabolism via a nitroreductase or glutathione conjugation mechanism:

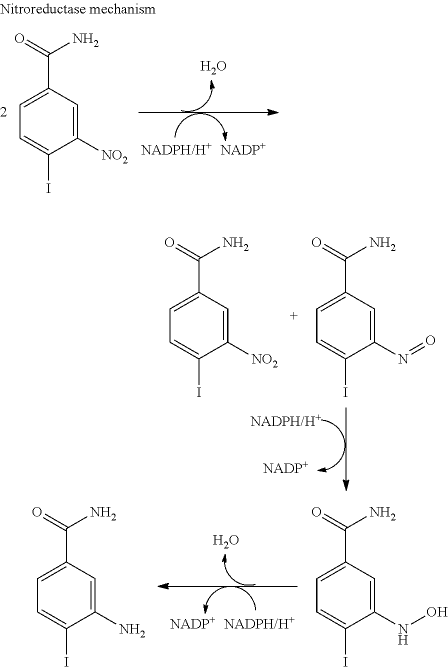

### 4-iodo-3-nitrobenzamide glutathione conjugation and metabolism

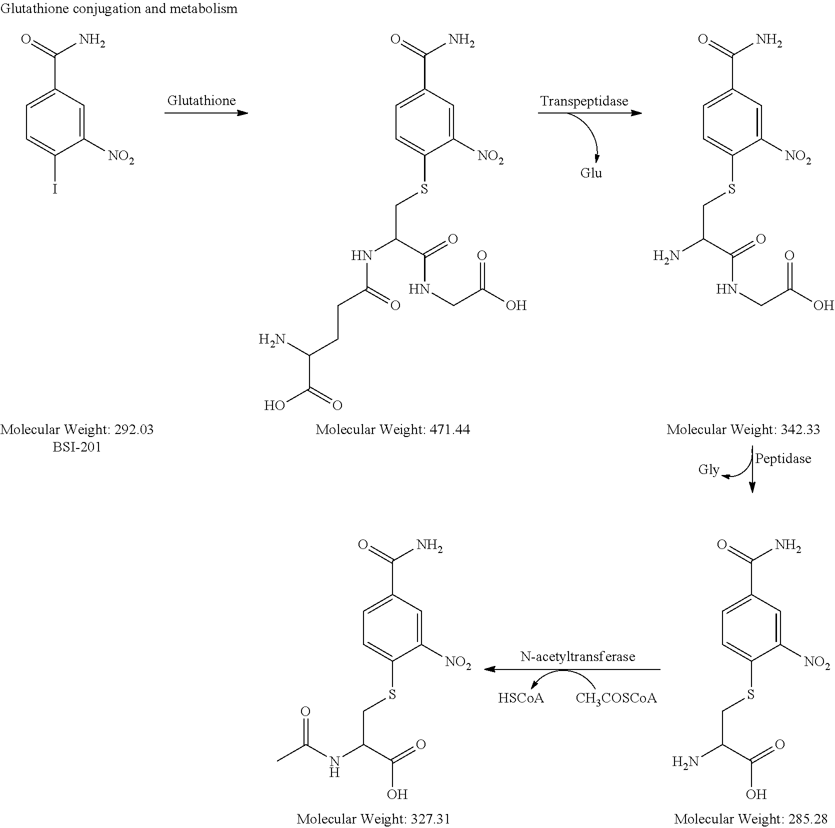

Any one of the metabolites of 4-iodo-3-nitrobenzamide described herein may be used in any one of the methods provided herein. Metabolites of 4-iodo-3-nitrobenzamide include, for example, 4-iodo-3-aminobenzoic acid (“IABA”), 4-iodo-3-aminobenzamide (“IABM”), 4-iodo-3-nitrosobenzamide (“BNO”), and 4-iodo-3-hydroxyaminobenzamide (“BNHOH”). Metabolites and methods of making metabolites are disclosed in U.S. Publication No. 2008/0103104 and U.S. Pat. No. 5,877,185, which are hereby incorporated by reference in their entirety, and in particular with respect to the metabolites and methods of making metabolites.

In some embodiments of any of the methods provided herein, 4-iodo-3-nitrobenzamide or a metabolite thereof or a pharmaceutically acceptable salt thereof is administered. In some embodiments, 4-iodo-3-nitrobenzamide or a pharmaceutically acceptable salt thereof is administered. In some embodiments, a metabolite of 4-iodo-3-nitrobenzamide is administered. In some embodiments, the metabolite of 4-iodo-3-nitrobenzamide is 4-iodo-3-aminobenzoic acid or 4-iodo-3-aminobenzamide.

The dosage range for the metabolites described herein used for treating squamous cell lung cancer may be in the range of about 0.0004 to about 0.5 mmol/kg (millimoles of metabolite per kilogram of individual's body weight), which dosage corresponds, on a molar basis, to a range of about 0.1 to about 100 mg/kg of 4-iodo-3-nitrobenzamide. Other effective ranges of dosages for metabolites are 0.0024-0.5 mmol/kg and 0.0048-0.25 mmol/kg. Such doses may be administered on a daily, every-other-daily, twice-weekly, weekly, bi-weekly, monthly or other suitable schedule. Essentially the same modes of administration may be employed for the metabolites as for 4-iodo-3-nitrobenzamide—e.g., oral, i.v., i.p., etc.

In some embodiments, 4-iodo-3-nitrobenzamide or a pharmaceutically acceptable salt thereof is administered. In some embodiments, a metabolite of 4-iodo-3-nitrobenzamide or a pharmaceutically acceptable salt of a metabolite of 4-iodo-3-nitrobenzamide is administered. The term “pharmaceutically acceptable salt” means those salts which retain the biological effectiveness and properties of the compounds used herein, and which are not biologically or otherwise undesirable. For example, a pharmaceutically acceptable salt does not interfere with the beneficial effect of the compound described herein in treating squamous cell lung cancer.

Typical salts are those of the inorganic ions, such as, for example, sodium, potassium, calcium and magnesium ions. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, where compounds contain a carboxy group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine. In some embodiments, 4-iodo-3-nitrobenzamide is formulated in 25% (w/v) hydroxypropyl-β-cyclodextrin and 10 mM phosphate buffer for intravenous administration as described in U.S. patent application Ser. No. 12/510,969, filed Jul. 28, 2009, with U.S. Publication No. US 2010/0160442, which is incorporated herein by reference.

### Gemcitabine

Gemcitabine (4-amino-1-[3,3-difluoro-4-hydroxy-5-(hydroxymethyl) tetrahydrofuran-2-yl]-1H-pyrimidin-2-one) (for example, it can be obtained as GEMZAR® from Eli Lilly and Company) is a nucleoside analog, which interferes with cellular division by blocking DNA synthesis, thus resulting in cell death, apparently through an apoptotic mechanism. Alternatively, gemcitabine is known as 4-amino-1-[(2R,4R,5R)-3,3-difluoro-4-hydroxy-5-(hydroxymethyl)tetrahydrofuran-2-yl]pyrimidin-2(1H)-one or 2′-deoxy-2′,2′-difluorocytidine. In some embodiments, gemcitabine used herein may include any pharmaceutically acceptable salt form (e.g., gemcitabine HCl or other salt forms). In some embodiments, gemcitabine is administered in any pharmaceutically acceptable formulation. The dosage of gemcitabine may be adjusted to the particular individual. In adults, the dosage of gemcitabine, when used in combination with 4-iodo-3-nitrobenzamide (or a metabolite thereof or a pharmaceutically acceptable salt thereof) and carboplatin is in the range of about 100 mg/m² to about 5000 mg/m², in the range of about 100 mg/m² to about 2000 mg/m², in the range of about 750 to about 1500 mg/m², about 900 to about 1400 mg/m², about 1000 mg/m², or about 1250 mg/m². The dimensions mg/m² refer to the amount of gemcitabine in milligrams (mg) per unit surface area of the individual in square meters (m²). Gemcitabine may be administered by intravenous (IV) infusion, e.g., over a period of about 10 to about 300 minutes, about 15 to about 180 minutes, about 20 to about 60 minutes, or about 30 minutes.

### Carboplatin

Carboplatin ((SP-4-2)-Diammine[1,1-cyclobutanedicarboxylato(2-)-O,O′ platinum) is a DNA alkylating agent. Alternatively, carboplatin is known as cis-Diammine(1,1-cyclobutanedicarboxylato)platinum(II). It is available, for example, from Bedford Laboratories. It is also marketed under the brand names Paraplatin® and Paraplatin-AQ. In some embodiments, carboplatin used herein may include any pharmaceutically acceptable salt form. In some embodiments, carboplatin is administered in any pharmaceutically acceptable formulation. The dosage of carboplatin is determined by calculating the area under the blood plasma concentration versus time curve (AUC) in mg/mL·minute by methods known to those skilled in the cancer chemotherapy art, taking into account the individual's renal activity estimated by measuring creatinine clearance or glomerular filtration rate. In some embodiments, the dosage of carboplatin for combination treatment along with gemcitabine and 4-iodo-3-nitrobenzamide (or a metabolite thereof or a pharmaceutically acceptable salt thereof) is calculated to provide an AUC of about 0.1-6 mg/ml·min, about 1-6 mg/ml·min, about 1.5 to about 5.5 mg/ml·min, about 3.5 to about 5.5 mg/ml·min or about 5 mg/ml·min (AUC 5, for example, is shorthand for 5 mg/ml·minute). Alternatively, the dosage of carboplatin is calculated based on the individual's body surface area. In some embodiments, a suitable dose of carboplatin is about 10 to about 800 mg/m², about 10 to about 400 mg/m², e.g., about any of 100 mg/m², 150 mg/m², 200 mg/m², 250 mg/m², 300 mg/m², 350 mg/m², 360 mg/m², or 400 mg/m². Carboplatin may be administered intravenously (IV) over a period of about 10 to about 300 minutes, about 30 to about 300 minutes, about 45 to about 250 minutes, about 60 minutes, about 120 minutes, about 150 minutes, about 180 minutes, about 210 minutes, about 240 minutes, about 270 minutes, or about 300 minutes.

### Anti-Tumor Agents

Any of the methods provided herein may further comprise administering at least one additional anti-tumor agent. For example, there are provided methods of treating squamous non-small cell lung cancer (“squamous cell lung cancer”) (e.g., stage IV squamous cell lung cancer) in an individual, comprising administering to the individual having squamous cell lung cancer an effective amount of: (i) 4-iodo-3-nitrobenzamide, a metabolite thereof, or a pharmaceutically acceptable salt thereof; (ii) gemcitabine; and (iii) carboplatin, wherein the method further comprises administering at least one anti-tumor agent to the individual. The anti-tumor agent may be an agent that is not (i) 4-iodo-3-nitrobenzamide, a metabolite thereof, or a pharmaceutically acceptable salt thereof; (ii) gemcitabine; or (iii) carboplatin.

Anti-tumor agents that may be used in the present invention include but are not limited to antitumor alkylating agents, antitumor antimetabolites, antitumor antibiotics, plant-derived antitumor agents, antitumor platinum-complex compounds, antitumor camptothecin derivatives, antitumor tyrosine kinase inhibitors, anti-tumor viral agent, monoclonal antibodies, interferons, biological response modifiers, and other agents that exhibit anti-tumor activities, or a pharmaceutically acceptable salt thereof.

In some embodiments, the anti-tumor agent is an alkylating agent. The term “alkylating agent” herein generally refers to an agent giving an alkyl group in the alkylation reaction in which a hydrogen atom of an organic compound is substituted with an alkyl group. Examples of anti-tumor alkylating agents include but are not limited to nitrogen mustard N-oxide, cyclophosphamide, ifosfamide, melphalan, busulfan, mitobronitol, carboquone, thiotepa, ranimustine, nimustine, temozolomide or carmustine.

In some embodiments, the anti-tumor agent is an antimetabolite. The term “antimetabolite” used herein includes, in a broad sense, substances which disturb normal metabolism and substances which inhibit the electron transfer system to prevent the production of energy-rich intermediates, due to their structural or functional similarities to metabolites that are important for living organisms (such as vitamins, coenzymes, amino acids and saccharides). Examples of antimetabolites that have anti-tumor activities include but are not limited to methotrexate, 6-mercaptopurine riboside, mercaptopurine, 5-fluorouracil, tegafur, doxifluridine, carmofur, cytarabine, cytarabine ocfosfate, enocitabine, S-1, gemcitabine, fludarabine or pemetrexed disodium, and preferred are 5-fluorouracil, S-1, gemcitabine and the like.

In some embodiments, the anti-tumor agent is an antitumor antibiotic. Examples of antitumor antibiotics include but are not limited to actinomycin D, doxorubicin, daunorubicin, neocarzinostatin, bleomycin, peplomycin, mitomycin C, aclarubicin, pirarubicin, epirubicin, zinostatin, stimalamer, idarubicin, sirolimus or valrubicin.

In some embodiments, the anti-tumor agent is a plant-derived antitumor agent. Examples of plant-derived antitumor agents include but are not limited to vincristine, vinblastine, vindesine, etoposide, sobuzoxane, docetaxel, paclitaxel and vinorelbine, and preferred are docetaxel and paclitaxel.

In some embodiments, the anti-tumor agent is a camptothecin derivative that exhibits anti-tumor activities. Examples of anti-tumor camptothecin derivatives include but are not limited to camptothecin, 10-hydroxycamptothecin, topotecan, irinotecan or 9-aminocamptothecin, with camptothecin, topotecan and irinotecan being preferred. Further, irinotecan is metabolized in vivo and exhibits antitumor effect as SN-38. The action mechanism and the activity of the camptothecin derivatives are believed to be virtually the same as those of camptothecin (e.g., Nitta et al., Gan to Kagaku Ryoho, 14, 850-857 (1987)).

In some embodiments, the anti-tumor agent is an organoplatinum compound or a platinum coordination compound having antitumor activity. The terms “organoplatinum compound,” “platinum compound,” or “platinum complex” and the like as used herein refer to a platinum-containing compound which provides platinum in ion form. Preferred organoplatinum compounds include but are not limited to cisplatin; cis-diamminediaquoplatinum (II)-ion; chloro(diethylenetriamine)-platinum (II) chloride; dichloro(ethylenediamine)-platinum (II); diammine(1,1-cyclobutanedicarboxylato) platinum (II) (carboplatin); spiroplatin; iproplatin; diammine(2-ethylmalonato)platinum (II); ethylenediaminemalonatoplatinum (II); aqua(1,2-diaminodicyclohexane)sulfatoplatinum (II); aqua(1,2-diaminodicyclohexane)malonatoplatinum (II); (1,2-diaminocyclohexane)malonatoplatinum (II); (4-carboxyphthalato)(1,2-diaminocyclohexane) platinum (II); (1,2-diaminocyclohexane)-(isocitrato)platinum (II); (1,2-diaminocyclohexane)oxalatoplatinum (II); ormaplatin; tetraplatin; carboplatin, nedaplatin and oxaliplatin, and preferred is carboplatin or oxaliplatin. Further, other antitumor organoplatinum compounds mentioned in the specification are known and are commercially available and/or producible by a person having ordinary skill in the art by conventional techniques.

In some embodiments, the anti-tumor agent is an antitumor tyrosine kinase inhibitor. The term “tyrosine kinase inhibitor” herein refers to a chemical substance inhibiting “tyrosine kinase” which transfers a λ-phosphate group of ATP to a hydroxyl group of a specific tyrosine in protein. Examples of anti-tumor tyrosine kinase inhibitors include but are not limited to gefitinib, imatinib, erlotinib, Sutent, Nexavar, Recentin, ABT-869, and Axitinib.

In some embodiments, the anti-tumor agent is an antibody or a binding portion of an antibody that exhibits anti-tumor activity. In some embodiments, the anti-tumor agent is a monoclonal antibody. Examples thereof include but are not limited to abciximab, adalimumab, alemtuzumab, basiliximab, bevacizumab, cetuximab, daclizumab, eculizumab, efalizumab, ibritumomab, tiuxetan, infliximab, muromonab-CD3, natalizumab, omalizumab, palivizumab, panitumumab, ranibizumab, gemtuzumab ozogamicin, rituximab, tositumomab, trastuzumab, or any antibody fragments specific for antigens.

In some embodiments, the anti-tumor agent is an interferon. Such interferon has antitumor activity, and it is a glycoprotein which is produced and secreted by most animal cells upon viral infection. It has not only the effect of inhibiting viral growth but also various immune effector mechanisms including inhibition of growth of cells (in particular, tumor cells) and enhancement of the natural killer cell activity, thus being designated as one type of cytokine. Examples of anti-tumor interferons include but are not limited to interferon α, interferon α-2a, interferon α-2b, interferon β, interferon γ-1a and interferon γ-n1.

In some embodiments, the anti-tumor agent is a biological response modifier. It is generally the generic term for substances or drugs for modifying the defense mechanisms of living organisms or biological responses such as survival, growth or differentiation of tissue cells in order to direct them to be useful for an individual against tumor, infection or other diseases. Examples of the biological response modifier include but are not limited to krestin, lentinan, sizofuran, picibanil and ubenimex.

In some embodiments, the anti-tumor agents include but are not limited to mitoxantrone, L-asparaginase, procarbazine, dacarbazine, hydroxycarbamide, pentostatin, tretinoin, alefacept, darbepoetin alfa, anastrozole, exemestane, bicalutamide, leuprorelin, flutamide, fulvestrant, pegaptanib octasodium, denileukin diftitox, aldesleukin, thyrotropin alfa, arsenic trioxide, bortezomib, capecitabine, and goserelin.

The above-described terms “antitumor alkylating agent”, “antitumor antimetabolite”, “antitumor antibiotic”, “plant-derived antitumor agent”, “antitumor platinum coordination compound”, “antitumor camptothecin derivative”, “antitumor tyrosine kinase inhibitor”, “monoclonal antibody”, “interferon”, “biological response modifier” and “other antitumor agent” are all known and are either commercially available or producible by a person skilled in the art by methods known per se or by well-known or conventional methods. The process for preparation of gefitinib is described, for example, in U.S. Pat. No. 5,770,599; the process for preparation of cetuximab is described, for example, in WO 96/40210; the process for preparation of bevacizumab is described, for example, in WO 94/10202; the process for preparation of oxaliplatin is described, for example, in U.S. Pat. Nos. 5,420,319 and 5,959,133; the process for preparation of gemcitabine is described, for example, in U.S. Pat. Nos. 5,434,254 and 5,223,608; and the process for preparation of camptothecin is described in U.S. Pat. Nos. 5,162,532, 5,247,089, 5,191,082, 5,200,524, 5,243,050 and 5,321,140; the process for preparation of irinotecan is described, for example, in U.S. Pat. No. 4,604,463; the process for preparation of topotecan is described, for example, in U.S. Pat. No. 5,734,056; the process for preparation of temozolomide is described, for example, in JP-B No. 4-5029; and the process for preparation of rituximab is described, for example, in JP-W No. 2-503143.

The above-mentioned antitumor alkylating agents are commercially available, as exemplified by the following: nitrogen mustard N-oxide from Mitsubishi Pharma Corp. as Nitrorin (tradename); cyclophosphamide from Shionogi & Co., Ltd. as Endoxan (tradename); ifosfamide from Shionogi & Co., Ltd. as Ifomide (tradename); melphalan from GlaxoSmithKline Corp. as Alkeran (tradename); busulfan from Takeda Pharmaceutical Co., Ltd. as Mablin (tradename); mitobronitol from Kyorin Pharmaceutical Co., Ltd. as Myebrol (tradename); carboquone from Sankyo Co., Ltd. as Esquinon (tradename); thiotepa from Sumitomo Pharmaceutical Co., Ltd. as Tespamin (tradename); ranimustine from Mitsubishi Pharma Corp. as Cymerin (tradename); nimustine from Sankyo Co., Ltd. as Nidran (tradename); temozolomide from Schering Corp. as Temodar (tradename); and carmustine from Guilford Pharmaceuticals Inc. as Gliadel Wafer (tradename).

The above-mentioned antitumor antimetabolites are commercially available, as exemplified by the following: methotrexate from Takeda Pharmaceutical Co., Ltd. as Methotrexate (tradename); 6-mercaptopurine riboside from Aventis Corp. as Thioinosine (tradename); mercaptopurine from Takeda Pharmaceutical Co., Ltd. as Leukerin (tradename); 5-fluorouracil from Kyowa Hakko Kogyo Co., Ltd. as 5-FU (tradename); tegafur from Taiho Pharmaceutical Co., Ltd. as Futraful (tradename); doxyfluridine from Nippon Roche Co., Ltd. as Furutulon (tradename); carmofur from Yamanouchi Pharmaceutical Co., Ltd. as Yamafur (tradename); cytarabine from Nippon Shinyaku Co., Ltd. as Cylocide (tradename); cytarabine ocfosfate from Nippon Kayaku Co., Ltd. as Strasid (tradename); enocitabine from Asahi Kasei Corp. as Sanrabin (tradename); S-1 from Taiho Pharmaceutical Co., Ltd. as TS-1 (tradename); gemcitabine from Eli Lilly & Co. as Gemzar (tradename); fludarabine from Nippon Schering Co., Ltd. as Fludara (tradename); and pemetrexed disodium from Eli Lilly & Co. as Alimta (tradename).

The above-mentioned antitumor antibiotics are commercially available, as exemplified by the following: actinomycin D from Banyu Pharmaceutical Co., Ltd. as Cosmegen (tradename); doxorubicin from Kyowa Hakko Kogyo Co., Ltd. as adriacin (tradename); daunorubicin from Meiji Seika Kaisha Ltd. as Daunomycin; neocarzinostatin from Yamanouchi Pharmaceutical Co., Ltd. as Neocarzinostatin (tradename); bleomycin from Nippon Kayaku Co., Ltd. as Bleo (tradename); pepromycin from Nippon Kayaku Co, Ltd. as Pepro (tradename); mitomycin C from Kyowa Hakko Kogyo Co., Ltd. as Mitomycin (tradename); aclarubicin from Yamanouchi Pharmaceutical Co., Ltd. as Aclacinon (tradename); pirarubicin from Nippon Kayaku Co., Ltd. as Pinorubicin (tradename); epirubicin from Pharmacia Corp. as Pharmorubicin (tradename); zinostatin stimalamer from Yamanouchi Pharmaceutical Co., Ltd. as Smancs (tradename); idarubicin from Pharmacia Corp. as Idamycin (tradename); sirolimus from Wyeth Corp. as Rapamune (tradename); and valrubicin from Anthra Pharmaceuticals Inc. as Valstar (tradename).

The above-mentioned plant-derived antitumor agents are commercially available, as exemplified by the following: vincristine from Shionogi & Co., Ltd. as Oncovin (tradename); vinblastine from Kyorin Pharmaceutical Co., Ltd. as Vinblastine (tradename); vindesine from Shionogi & Co., Ltd. as Fildesin (tradename); etoposide from Nippon Kayaku Co., Ltd. as Lastet (tradename); sobuzoxane from Zenyaku Kogyo Co., Ltd. as Perazolin (tradename); docetaxel from Aventis Corp. as Taxsotere (tradename); paclitaxel from Bristol-Myers Squibb Co. as Taxol (tradename); and vinorelbine from Kyowa Hakko Kogyo Co., Ltd. as Navelbine (tradename).

The above-mentioned antitumor platinum coordination compounds are commercially available, as exemplified by the following: cisplatin from Nippon Kayaku Co., Ltd. as Randa (tradename); carboplatin from Bristol-Myers Squibb Co. as Paraplatin (tradename); nedaplatin from Shionogi & Co., Ltd. as Aqupla (tradename); and oxaliplatin from Sanofi-Synthelabo Co. as Eloxatin (tradename).

The above-mentioned antitumor camptothecin derivatives are commercially available, as exemplified by the following: irinotecan from Yakult Honsha Co., Ltd. as Campto (tradename); topotecan from GlaxoSmithKline Corp. as Hycamtin (tradename); and camptothecin from Aldrich Chemical Co., Inc., U.S.A.

The above-mentioned antitumor tyrosine kinase inhibitors are commercially available, as exemplified by the following: gefitinib from AstraZeneca Corp. as Iressa (tradename); imatinib from Novartis AG as Gleevec (tradename); and erlotinib from OSI Pharmaceuticals Inc. as Tarceva (tradename).

The above-mentioned monoclonal antibodies are commercially available, as exemplified by the following: cetuximab from Bristol-Myers Squibb Co. as Erbitux (tradename); bevacizumab from Genentech, Inc. as Avastin (tradename); rituximab from Biogen Idec Inc. as Rituxan (tradename); alemtuzumab from Berlex Inc. as Campath (tradename); and trastuzumab from Chugai Pharmaceutical Co., Ltd. as Herceptin (tradename).

The above-mentioned interferons are commercially available, as exemplified by the following: interferon α from Sumitomo Pharmaceutical Co., Ltd. as Sumiferon (tradename); interferon α-2a from Takeda Pharmaceutical Co., Ltd. as Canferon-A (tradename); interferon α-2b from Schering-Plough Corp. as Intron A (tradename); interferon β from Mochida Pharmaceutical Co., Ltd. as IFN.beta. (tradename); interferon γ-1a from Shionogi & Co., Ltd. as Immunomax-γ (tradename); and interferon γ-n1 from Otsuka Pharmaceutical Co., Ltd. as Ogamma (tradename).

The above-mentioned biological response modifiers are commercially available, as exemplified by the following: krestin from Sankyo Co., Ltd. as krestin (tradename); lentinan from Aventis Corp. as Lentinan (tradename); sizofuran from Kaken Seiyaku Co., Ltd. as Sonifuran (tradename); picibanil from Chugai Pharmaceutical Co., Ltd. as Picibanil (tradename); and ubenimex from Nippon Kayaku Co., Ltd. as Bestatin (tradename).

The above-mentioned other antitumor agents are commercially available, as exemplified by the following: mitoxantrone from Wyeth Lederle Japan, Ltd. as Novantrone (tradename); L-asparaginase from Kyowa Hakko Kogyo Co., Ltd. as Leunase (tradename); procarbazine from Nippon Roche Co., Ltd. as Natulan (tradename); dacarbazine from Kyowa Hakko Kogyo Co., Ltd. as Dacarbazine (tradename); hydroxycarbamide from Bristol-Myers Squibb Co. as Hydrea (tradename); pentostatin from Kagaku Oyobi Kessei Ryoho Kenkyusho as Coforin (tradename); tretinoin from Nippon Roche Co., Ltd. As Vesanoid (tradename); alefacept from Biogen Idec Inc. as Amevive (tradename); darbepoetin alfa from Amgen Inc. as Aranesp (tradename); anastrozole from AstraZeneca Corp. as Arimidex (tradename); exemestane from Pfizer Inc. as Aromasin (tradename); bicalutamide from AstraZeneca Corp. as Casodex (tradename); leuprorelin from Takeda Pharmaceutical Co., Ltd. as Leuplin (tradename); flutamide from Schering-Plough Corp. as Eulexin (tradename); fulvestrant from AstraZeneca Corp. as Faslodex (tradename); pegaptanib octasodium from Gilead Sciences, Inc. as Macugen (tradename); denileukin diftitox from Ligand Pharmaceuticals Inc. as Ontak (tradename); aldesleukin from Chiron Corp. as Proleukin (tradename); thyrotropin alfa from Genzyme Corp. as Thyrogen (tradename); arsenic trioxide from Cell Therapeutics, Inc. as Trisenox (tradename); bortezomib from Millennium Pharmaceuticals, Inc. as Velcade (tradename); capecitabine from Hoffmann-La Roche, Ltd. as Xeloda (tradename); and goserelin from AstraZeneca Corp. as Zoladex (tradename). The term “antitumor agent” as used in the specification includes the above-described antitumor alkylating agent, antitumor antimetabolite, antitumor antibiotic, plant-derived antitumor agent, antitumor platinum coordination compound, antitumor camptothecin derivative, antitumor tyrosine kinase inhibitor, monoclonal antibody, interferon, biological response modifier, and other antitumor agents.

Other anti-tumor agents or anti-neoplastic agents can be used in combination with benzopyrone compounds. Other anti-tumor agents or anti-neoplastic agents can also be used in combination with (i) 4-iodo-3-nitrobenzamide, a metabolite thereof, or a pharmaceutically acceptable salt thereof; (ii) gemcitabine; and (iii) carboplatin. Such suitable anti-tumor agents or anti-neoplastic agents include, but are not limited to, 13-cis-Retinoic Acid, 2-CdA, 2-Chlorodeoxyadenosine, 5-Azacitidine, 5-Fluorouracil, 5-FU, 6-Mercaptopurine, 6-MP, 6-TG, 6-Thioguanine, Abraxane, Accutane, Actinomycin-D, Adriamycin, Adrucil, Agrylin, Ala-Cort, Aldesleukin, Alemtuzumab, ALIMTA, Alitretinoin, Alkaban-AQ, Alkeran, All-transretinoic Acid, Alpha Interferon, Altretamine, Amethopterin, Amifostine, Aminoglutethimide, Anagrelide, Anandron, Anastrozole, Arabinosylcytosine, Ara-C, Aranesp, Aredia, Arimidex, Aromasin, Arranon, Arsenic Trioxide, Asparaginase, ATRA, Avastin, Azacitidine, BCG, BCNU, Bendamustine, Bevacizumab, Bexarotene, BEXXAR, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine Wafer, Casodex, CC-5013, CCI-779, CCNU, CDDP, CeeNU, Cerubidine, Cetuximab, Chlorambucil, Cisplatin, Citrovorum Factor, Cladribine, Cortisone, Cosmegen, CPT-11, Cyclophosphamide, Cytadren, Cytarabine, Cytarabine Liposomal, Cytosar-U, Cytoxan, Dacarbazine, Dacogen, Dactinomycin, Darbepoetin Alfa, Dasatinib, Daunomycin, Daunorubicin, Daunorubicin Hydrochloride, Daunorubicin Liposomal, DaunoXome, Decadron, Decitabine, Delta-Cortef, Deltasone, Denileukin Diftitox, DepoCyt™, Dexamethasone, Dexamethasone Acetate, Dexamethasone Sodium Phosphate, Dexasone, Dexrazoxane, DHAD, DIC, Diodex, Docetaxel, Doxil, Doxorubicin, Doxorubicin Liposomal, Droxia™, DTIC, DTIC-Dome, Duralone, Efudex, Eligard, Ellence, Eloxatin, Elspar, Emcyt, Epirubicin, Epoetin Alfa, Erbitux, Erlotinib, Erwinia L-asparaginase, Estramustine, Ethyol, Etopophos, Etoposide, Etoposide Phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar & Gemzar Side Effects—Chemotherapy Drugs, Gleevec, Gliadel Wafer, GM-CSF, Goserelin, Granulocyte—Colony Stimulating Factor, Granulocyte Macrophage Colony Stimulating Factor, Halotestin, Herceptin, Hexadrol, Hexylen, Hexamethylmelamine, HMM, Hycamtin, Hydrea, Hydrocort Acetate, Hydrocortisone, Hydrocortisone Sodium Phosphate, Hydrocortisone Sodium Succinate, Hydrocortone Phosphate, Hydroxyurea, Ibritumomab, Ibritumomab Tiuxetan, Idamycin, Idarubicin, Ifex, IFN-alpha, Ifosfamide, IL-11, IL-2, Imatinib mesylate, Imidazole Carboxamide, Interferon alfa, Interferon Alfa-2b (PEG Conjugate), Interleukin-2, Interleukin-11, Intron A (interferon alfa-2b), Iressa, Irinotecan, Isotretinoin, Ixabepilone, Ixempra, Kidrolase (t), Lanacort, Lapatinib, L-asparaginase, LCR, Lenalidomide, Letrozole, Leucovorin, Leukeran, Leukine, Leuprolide, Leurocristine, Leustatin, Liposomal Ara-C, Liquid Pred, Lomustine, L-PAM, L-Sarcolysin, Lupron, Lupron Depot, Matulane, Maxidex, Mechlorethamine, Mechlorethamine Hydrochloride, Medralone, Medrol, Megace, Megestrol, Megestrol Acetate, Melphalan, Mercaptopurine, Mesna, Mesnex, Methotrexate, Methotrexate Sodium, Methylprednisolone, Meticorten, Mitomycin, Mitomycin-C, Mitoxantrone, M-Prednisol, MTC, MTX, Mustargen, Mustine, Mutamycin, Myleran, Mylocel, Mylotarg, Navelbine, Nelarabine, Neosar, Neulasta, Neumega, Neupogen, Nexavar, Nilandron, Nilutamide, Nipent, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Paclitaxel Protein-bound, Pamidronate, Panitumumab, Panretin, Paraplatin, Pediapred, PEG Interferon, Pegaspargase, Pegfilgrastim, PEG-INTRON, PEG-L-asparaginase, PEMETREXED, Pentostatin, Phenylalanine Mustard, Platinol, Platinol-AQ, Prednisolone, Prednisone, Prelone, Procarbazine, PROCRIT, Proleukin, Prolifeprospan 20 with Carmustine Implant, Purinethol, Raloxifene, Revlimid, Rheumatrex, Rituxan, Rituximab, Roferon-A (Interferon Alfa-2a), Rubex, Rubidomycin hydrochloride, Sandostatin, Sandostatin LAR, Sargramostim, Solu-Cortef, Solu-Medrol, Sorafenib, SPRYCEL, STI-571, Streptozocin, SU11248, Sunitinib, Sutent, Tamoxifen, Tarceva, Targretin, Taxol, Taxotere, Temodar, Temozolomide, Temsirolimus, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Torisel, Tositumomab, Trastuzumab, Tretinoin, Trexall™, Trisenox, TSPA, TYKERB, VCR, Vectibix, Vectibix, Velban, Velcade, VePesid, Vesanoid, Viadur, Vidaza, Vinblastine, Vinblastine Sulfate, Vincasar Pfs, Vincristine, Vinorelbine, Vinorelbine tartrate, VLB, VM-26, Vorinostat, VP-16, Vumon, Xeloda, Zanosar, Zevalin, Zinecard, Zoladex, Zoledronic acid, Zolinza, Zometa.

### Treatment of Squamous Cell Lung Cancer

Provided herein are methods of treating squamous non-small cell lung cancer (“squamous cell lung cancer”) (e.g., stage IV squamous cell lung cancer) in an individual, comprising administering to the individual having squamous cell lung cancer an effective amount of: (i) 4-iodo-3-nitrobenzamide, a metabolite thereof, or a pharmaceutically acceptable salt thereof; (ii) gemcitabine; and (iii) carboplatin. In some embodiments, the individual has squamous cell bronchogenic carcinoma.

In some embodiments, the squamous cell lung cancer is any of stage 0, I, II, III, or IV. In some embodiments, the squamous cell lung cancer is any of stage IA, IB, IIA, IIB, IIIA, IIIB, or IV. In some embodiments, the squamous cell lung cancer is stage IV. For example, there is provided a method of treating stage IV squamous cell lung cancer in an individual, comprising administering to the individual having squamous cell lung cancer an effective amount of: (i) 4-iodo-3-nitrobenzamide, a metabolite thereof, or a pharmaceutically acceptable salt thereof; (ii) gemcitabine; and (iii) carboplatin.

In some embodiments of any one of the methods provided herein, the squamous cell lung cancer is metastatic. In some embodiments of any one of the methods provided herein, the squamous cell lung cancer is not metastatic. In some embodiments, the squamous cell lung cancer is locally advanced. In some embodiments, the individual has distant metastases. In some embodiments, the individual has distant metastases in extrathoracic organ(s). In some embodiments, the individual does not have distant metastases. In some embodiments, the individual has disseminated metastases. In some embodiments, the individual has malignant pleural or pericardial effusion. In some embodiments, the individual has separate lung tumor nodule(s) in a contralateral lobe. In some embodiments, the individual has lung tumor with pleural nodules. In some embodiments, the squamous cell lung cancer is recurrent. In some embodiments, the squamous cell lung cancer is resectable. In some embodiments, the squamous cell lung cancer is newly diagnosed. In some embodiments, the squamous cell lung cancer has not been previously treated. In some embodiments, the individual has newly diagnosed stage IV squamous cell lung cancer. In some embodiments, the individual has stage IV squamous cell lung cancer at diagnosis or recurrent after resection of the squamous cell lung cancer. In some embodiments, the individual has previously received adjuvant chemotherapy for early stage lung cancer. In some embodiments, the individual has not previously received adjuvant chemotherapy for early stage lung cancer. In some embodiments, the individual has not previously received systemic treatment for advanced squamous cell lung cancer. In some embodiments, the individual has previously received adjuvant therapy for early stage lung cancer and the adjuvant therapy ended at least 12 months ago. In some embodiments, the individual has not previously received a therapy comprising chemotherapy and radiation therapy for locally advanced squamous cell lung cancer. Other characteristics of individuals that can be treated with any one of the methods provided herein are provided in Examples 1-3 of the present disclosure. In some embodiments, the individual having squamous cell lung cancer has a history of smoking. In some embodiments, the individual having squamous cell lung cancer does not have a history of smoking.

An individual treated by a method described herein may have one or more of the following: (1) Newly diagnosed, stage IV squamous cell lung cancer (this includes individuals who present with disseminated metastases, and those with a malignant pleural or pericardial effusion (e.g., formerly stage IIIB in the 6th TNM staging system)); (2) Stage IV at diagnosis or recurrent after resection±adjuvant chemotherapy for early stage disease (AJCC Staging System 7th edition); (3) No previous systemic treatment for advanced NSCLC; (4) Individuals who have received prior adjuvant therapy for early-stage lung cancer are eligible if at least 12 months have elapsed from that treatment; (5) Histologically confirmed squamous cell bronchogenic carcinoma (individuals whose tumors contain mixed non-small cell histologies are eligible, as long as squamous carcinoma is the predominant histology) (mixed tumors with small cell anaplastic elements may not be eligible for a treatment described herein); (6) Individuals with previous radiotherapy as definitive therapy for locally advanced non-small cell lung cancer are eligible, as long as the recurrence is outside the original radiation therapy port (radiation therapy must have been completed >4 weeks prior to the initiation of study treatment; individuals who have received palliative radiation therapy for symptomatic metastases must have completed treatment >14 days prior the initiation of the study treatment) (individuals who have received chemo/radiation for locally advanced NSCLC may not be eligible for a treatment described herein); (7) Presence of evaluable (measureable or non-measurable) disease; (8) ECOG performance status of 0 or 1. An individual treated by a method described herein may not have one or more of the following: (1) Prior treatment with gemcitabine, carboplatin (except in the adjuvant setting), or 4-iodo-3-nitrobenzamide; (2) Past or current history of neoplasm other than the entry diagnosis, with the exception of treated non-melanoma skin cancer or carcinoma in-situ of any primary site, or invasive cancers treated definitively, with treatment ending >5 years previously and no evidence of recurrences; (3) Active brain metastases (individuals with treated brain metastases may be eligible for a treatment described herein, if (a) radiation therapy was completed at least 2 weeks prior to study entry; (b) follow-up scan shows no disease progression; and (c) individual does not require steroids); (4) Prior combined modality treatment (chemotherapy+radiation therapy) for locally advanced NSCLC.

Staging of lung cancer (e.g., squamous cell lung cancer) is known to one of ordinary skill in the art. Staging may be according to any of the methods known in the art. For example, the staging of lung cancer may be according to the staging criteria set forth by American Joint Committee on Cancer (“AJCC”), for example, according to AJCC Cancer Staging Manual (e.g., 7th edition). The lung cancer staging may be according to the AJCC 7th edition for lung cancer, available at http://www.cancerstaging.org/staging/posters/lung12×15.pdf (last accessed on Mar. 4, 2011), the content of which is incorporated herein by reference in its entirety. The staging may be assessed by, for example, thoracic CT, PET scan, brain MRI.

Staging of lung cancer (e.g., squamous cell lung cancer) can be according to the TNM classification. The UICC and the AJCC initially introduced this system in 1972 with staging and end-results reporting. This scheme has been modified and refined over the years. They are formulated together but appear in separate books—namely, the UICC TNM Classification of Malignant Tumors and the AJCC Cancer Staging Manual. TNM is a dual system with a pretreatment Clinical classification (cTNM or TNM) and a postsurgical histopathologic Pathological classification (pTNM). The TNM staging system takes into account the degree of spread of the primary tumor, represented by T; the extent of regional lymph node involvement, represented by N; and the presence or absence of distant metastases, represented by M. The TNM system is used for all lung carcinomas except SCLCs, which are staged separately. In the TNM systems, 4 stages are further subdivided into I-III and A or B subtypes.

The definitions of T, M, N regarding staging of lung cancer may be in accordance with the following:

Primary Tumor (“T”):

TX: Primary tumor cannot be assessed, or tumor proven by the presence of malignant cells in sputum or bronchial washings but not visualized by imaging or bronchoscopy; T0: No evidence of primary tumor; T is: Carcinoma in situ; T1: Tumor 3 cm or less in greatest dimension, surrounded by lung or visceral pleura, without bronchoscopic evidence of invasion more proximal than the lobar bronchus (for example, not in the main bronchus) (the uncommon superficial spreading tumor of any size with its invasive component limited to the bronchial wall, which may extend proximally to the main bronchus, is classified as T1a); T1a: Tumor 2 cm or less in greatest dimension; T1b: Tumor more than 2 cm but 3 cm or less in greatest dimension; T2: Tumor more than 3 cm but 7 cm or less or tumor with any of the following features (T2 tumors with these features are classified T2a if 5 cm or less): involves main bronchus, 2 cm or more distal to the carina; invades visceral pleura (PL1 or PL2); associated with atelectasis or obstructive pneumonitis that extends to the hilar region but does not involve the entire lung; T2a: Tumor more than 3 cm but 5 cm or less in greatest dimension; T2b: Tumor more than 5 cm but 7 cm or less in greatest dimension; T3: Tumor more than 7 cm or one that directly invades any of the following: parietal pleural (PL3), chest wall (including superior sulcus tumors), diaphragm, phrenic nerve, mediastinal pleura, parietal pericardium; or tumor in the main bronchus less than 2 cm distal to the carinal but without involvement of the carina; or associated atelectasis or obstructive pneumonitis of the entire lung or separate tumor nodule(s) in the same lobe; T4: Tumor of any size that invades any of the following: mediastinum, heart, great vessels, trachea, recurrent laryngeal nerve, esophagus, vertebral body, carina, separate tumor nodule(s) in a different ipsilateral lobe.

Distant Metastasis (“M”):

M0: No distant metastasis; M1: Distant metastasis; M1a: Separate tumor nodule(s) in a contralateral lobe, tumor with pleural nodules or malignant pleural (or pericardial) effusion (most pleural (and pericardial) effusions with lung cancer are due to tumor. In a few patients, however, multiple cytopathologic examinations of pleural (pericardial) fluid are negative for tumor, and the fluid is nonbloody and is not an exudate. Where these elements and clinical judgment dictate that the effusion is not related to the tumor, the effusion should be excluded as a staging element and the patient should be classified as M0); M1b: Distant metastasis (in extrathoracic organs).

Regional Lymph Nodes (“N”):

NX: Regional lymph nodes cannot be assessed; N0: No regional lymph node metastases; N1: Metastasis in ipsilateral peribronchial and/or ipsilateral hilar lymph nodes and intrapulmonary nodes, including involvement by direct extension; N2: Metastasis in ipsilateral mediastinal and/or subcarinal lymph node(s); N3: Metastasis in contralateral mediastinal, contralateral hilar, ipsilateral or contralateral scalene, or supraclavicular lymph node(s).

The staging of lung cancer may be in accordance with the following table.

ANATOMIC STAGE/PROGNOSTIC GROUPS

| | | | |
|---|---|---|---|
| Occult Carcinoma | TX | N0 | M0 |
| Stage 0 | Tis | N0 | M0 |
| Stage IA | T1a | N0 | M0 |
| | T1b | N0 | M0 |
| Stage IB | T2a | N0 | M0 |
| Stage IIA | T2b | N0 | M0 |
| | T1a | N1 | M0 |
| | T1b | N1 | M0 |
| | T2a | N1 | M0 |
| Stage IIB | T2b | N1 | M0 |
| | T3 | N0 | M0 |
| Stage IIIA | T1a | N2 | M0 |
| | T1b | N2 | M0 |
| | T2a | N2 | M0 |
| | T2b | N2 | M0 |
| | T3 | N1 | M0 |
| | T3 | N2 | M0 |
| | T4 | N0 | M0 |
| | T4 | N1 | M0 |
| Stage IIIB | T1a | N3 | M0 |
| | T1b | N3 | M0 |
| | T2a | N3 | M0 |
| | T2b | N3 | M0 |
| | T3 | N3 | M0 |
| | T4 | N2 | M0 |
| | T4 | N3 | M0 |
| Stage IV | Any T | Any N | M1a |
| | Any T | Any N | M1b |

In some embodiments of any one of the methods provided herein, at least one therapeutic effect is obtained, said at least one therapeutic effect being reduction in size of a tumor, reduction in metastasis, complete remission, partial remission, pathologic complete response, increase in overall response rate, or stable disease. In some embodiments, a comparable clinical benefit rate (CBR=CR+PR+SD≧6 months) is obtained with treatment of the 4-iodo-3-nitrobenzamide (or a metabolite thereof, or a pharmaceutically acceptable salt thereof) in combination with gemcitabine and carboplatin as compared to treatment with gemcitabine and carboplatin without 4-iodo-3-nitrobenzamide (or a metabolite thereof, or a pharmaceutically acceptable salt thereof). In some embodiments, the improvement of clinical benefit rate is at least about 20%, 30%, 40%, 50%, 60%, 70%, 80% or more.

In some embodiments of any one of the methods provided herein, the administration of 4-iodo-3-nitrobenzamide (or a metabolite thereof, or a pharmaceutically acceptable salt thereof) in combination with gemcitabine and carboplatin results in a complete response, partial response, or stable disease. Any one of the methods provided herein may result in increase in overall response rate, increase in overall survival, or increase in progression free survival.

In some embodiments, the individual has measurable disease. Measurable disease may be determined by RECIST version 1.1 criteria, which is described in Eisenhauer E A et al. 2009, Eur J Cancer., 45(2):228-47, the disclosure of which is incorporated by reference in its entirety. For example, tumor lesions/lymph nodes may be categorized measurable as follows: (1) for tumor lesions: must be accurately measured in at least one dimension (longest diameter in the plane of measurement is to be recorded) with a minimum size of: (i) 10 mm by CT scan (CT scan slice thickness no greater than 5 mm), (ii) 10 mm caliper measurement by clinical exam (lesions which cannot be accurately measured with calipers should be recorded as non-measurable), (iii) 20 mm by chest X-ray; (2) for malignant lymph nodes: to be considered pathologically enlarged and measurable, a lymph node must be ≧15 mm in short axis when assessed by CT scan (CT scan slice thickness recommended to be no greater than 5 mm). Non-measurable tumor may include all other lesions, including small lesions (longest diameter <10 mm or pathological lymph nodes with P10 to <15 mm short axis) as well as truly non-measurable lesions. Lesions considered truly non-measurable may include: leptomeningeal disease, ascites, pleural or pericardial effusion, inflammatory breast disease, lymphangitic involvement of skin or lung, abdominal masses/abdominal organomegaly identified by physical exam that is not measurable by reproducible imaging techniques.

Response rates may be determined according to RECIST version 1.1 criteria. For example, regarding target lesion, complete Response (CR) may be defined as disappearance of all target lesions; any pathological lymph nodes (whether target or nontarget) must have reduction in short axis to <10 mm. Regarding target lesion, partial response (PR) may be defined as at least a 30% decrease in the sum of diameters of target lesions, taking as reference the baseline sum diameters. Regarding target lesion, progressive disease (PD) may be defined as at least a 20% increase in the sum of diameters of target lesions, taking as reference the smallest sum on study (this includes the baseline sum if that is the smallest on study). In addition to the relative increase of 20%, the sum may also demonstrate an absolute increase of at least 5 mm. Regarding target lesion, stable disease (SD) may be defined as neither sufficient shrinkage to qualify for PR nor sufficient increase to qualify for PD. Regarding non-target lesion, CR may be disappearance of all non-target lesions and normalisation of tumour marker level (all lymph nodes may be non-pathological in size (<10 mm short axis)). Regarding non-target lesion, non-CR/non-PD may be persistence of one or more non-target lesion(s) and/or maintenance of tumour marker level above the normal limits. Regarding non-target lesion, Progressive Disease (PD) may be unequivocal progression of existing non-target lesions.

Efficacy parameters may be determined by any of methods known to one skilled in the art. For example, they may be determined according to RECIST version 1.1 criteria. For example, objective response rate may be defined as the proportion of individuals with a best overall response of complete response or partial response. Progression-free survival may be defined as the time from the date of randomization until the date of first observation of disease progression, or the date of death. Overall survival may be defined as the time from the date of randomization until the date of death.

In some embodiments of any one of the methods provided herein, the individual has not received prior chemotherapy (e.g., prior chemotherapy for lung cancer such as squamous cell lung cancer). In some embodiments, the individual has received prior chemotherapy (e.g., prior chemotherapy for lung cancer such as squamous cell lung cancer). In some embodiments, the individual has not received prior chemotherapy comprising 4-iodo-3-nitrobenzamide, a metabolite thereof, or a pharmaceutically acceptable salt thereof. In some embodiments, the individual has not received prior chemotherapy comprising gemcitabine. In some embodiments, the individual has not received prior chemotherapy comprising carboplatin. In some embodiments, the individual has received prior chemotherapy comprising carboplatin in the adjuvant setting. In some embodiments, the individual has received carboplatin in the adjuvant setting for lung cancer. In some embodiments, the individual has received carboplatin in the adjuvant setting for early stage lung cancer. In some embodiments, the lung cancer described herein is squamous cell lung cancer.

### Combination Therapies

In some embodiments of any one of the methods provided herein, the method further comprises surgery, radiation therapy, chemotherapy, gene therapy, DNA therapy, adjuvant therapy, neoadjuvant therapy, RNA therapy, viral therapy, immunotherapy, nanotherapy or a combination thereof.

In some embodiments, there is provided a method of treating squamous non-small cell lung cancer (“squamous cell lung cancer”) (e.g., stage IV squamous cell lung cancer) in an individual, comprising administering to the individual having squamous cell lung cancer an effective amount of: (i) 4-iodo-3-nitrobenzamide, a metabolite thereof, or a pharmaceutically acceptable salt thereof; (ii) gemcitabine; and (iii) carboplatin, wherein the method further comprises one or more additional therapy or therapies used in the treatment of the squamous cell lung cancer described herein. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments the additional therapy is surgery. In some embodiments, the additional therapy is a combination of radiation therapy and surgery. In some embodiments, the additional therapy is gamma irradiation.

Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, by a significant period of time.

### Radiation Therapy

Radiation therapy (or radiotherapy) is the medical use of ionizing radiation as part of cancer treatment to control malignant cells. Radiotherapy may be used for curative or adjuvant cancer treatment. It is used as palliative treatment (where cure is not possible and the aim is for local disease control or symptomatic relief) or as therapeutic treatment (where the therapy has survival benefit and it can be curative). Radiotherapy is used for the treatment of malignant tumors and may be used as the primary therapy. It is also common to combine radiotherapy with surgery, chemotherapy, hormone therapy or some mixture of the three. Most common cancer types can be treated with radiotherapy in some way. The precise treatment intent (curative, adjuvant, neoadjuvant, therapeutic, or palliative) will depend on the tumour type, location, and stage, as well as the general health of the individual.

Radiation therapy is commonly applied to the cancerous tumor. The radiation fields may also include the draining lymph nodes if they are clinically or radiologically involved with tumor, or if there is thought to be a risk of subclinical malignant spread. It is necessary to include a margin of normal tissue around the tumor to allow for uncertainties in daily set-up and internal tumor motion.

Radiation therapy works by damaging the DNA of cells. The damage is caused by a photon, electron, proton, neutron, or ion beam directly or indirectly ionizing the atoms which make up the DNA chain. Indirect ionization happens as a result of the ionization of water, forming free radicals, notably hydroxyl radicals, which then damage the DNA. In the most common forms of radiation therapy, most of the radiation effect is through free radicals. Because cells have mechanisms for repairing DNA damage, breaking the DNA on both strands proves to be the most significant technique in modifying cell characteristics. Because cancer cells generally are undifferentiated and stem cell-like, they reproduce more, and have a diminished ability to repair sub-lethal damage compared to most healthy differentiated cells. The DNA damage is inherited through cell division, accumulating damage to the cancer cells, causing them to die or reproduce more slowly. Proton radiotherapy works by sending protons with varying kinetic energy to precisely stop at the tumor.

Gamma rays are also used to treat some types of cancer including lung cancer. In the procedure called gamma-knife surgery, multiple concentrated beams of gamma rays are directed on the growth in order to kill the cancerous cells. The beams are aimed from different angles to focus the radiation on the growth while minimizing damage to the surrounding tissues.

Radiosensitizers are known to increase the sensitivity of cancerous cells to the toxic effects of electromagnetic radiation. Many cancer treatment protocols currently employ radiosensitizers activated by the electromagnetic radiation of x-rays. Examples of x-ray activated radiosensitizers include, but are not limited to, the following: metronidazole, misonidazole, desmethylmisonidazole, pimonidazole, etanidazole, nimorazole, mitomycin C, RSU 1069, SR 4233, E09, RB 6145, nicotinamide, 5-bromodeoxyuridine (BUdR), 5-iododeoxyuridine (IUdR), bromodeoxycytidine, fluorodeoxyuridine (FudR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, photofrin, benzoporphyrin derivatives, NPe6, tin etioporphyrin SnET2, pheoborbide-alpha, bacteriochlorophyll-alpha, naphthalocyanines, phthalocyanines, zinc phthalocyanine, and therapeutically effective analogs and derivatives of the same.

### Gene Therapy Agents

Gene therapy agents insert copies of genes into a specific set of a individual's cells, and can target both cancer and non-cancer cells. The goal of gene therapy can be to replace altered genes with functional genes, to stimulate an individual's immune response to cancer, to make cancer cells more sensitive to chemotherapy, to place “suicide” genes into cancer cells, or to inhibit angiogenesis. Genes may be delivered to target cells using viruses, liposomes, or other carriers or vectors. This may be done by injecting the gene-carrier composition into the individual directly, or ex vivo, with infected cells being introduced back into a individual. Such compositions are suitable for use in the present invention.

### Adjuvant Therapy

Adjuvant therapy is a treatment given after the primary treatment to increase the chances of a cure. Adjuvant therapy may include chemotherapy, radiation therapy, hormone therapy, or biological therapy.

Because the principal purpose of adjuvant therapy is to kill any cancer cells that may have spread, treatment is usually systemic (uses substances that travel through the bloodstream, reaching and affecting cancer cells all over the body). For example, adjuvant therapy for lung cancer involves chemotherapy or hormone therapy, either alone or in combination.

Adjuvant chemotherapy is the use of drugs to kill cancer cells. For example, research has shown that using chemotherapy as adjuvant therapy for early stage lung cancer helps to prevent the original cancer from returning. Adjuvant chemotherapy is usually a combination of anticancer drugs, which has been shown to be more effective than a single anticancer drug.

Radiation therapy is sometimes used as a local adjuvant treatment. Radiation therapy is considered adjuvant treatment when it is given before or after surgical treatment, e.g., a mastectomy. Such treatment is intended to destroy cancer cells that have spread to nearby parts of the body, such as the chest wall or lymph nodes.

### Neoadjuvant Therapy

Neoadjuvant therapy refers to a treatment given before the primary treatment. Examples of neoadjuvant therapy include chemotherapy, radiation therapy, and hormone therapy. In treating squamous lung cancer, neoadjuvant therapy refers to a treatment given before the primary surgical treatment. Examples of neoadjuvant therapy include chemotherapy and radiation therapy.

### Oncolytic Viral Therapy

Viral therapy for cancer utilizes a type of viruses called oncolytic viruses. An oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site.

There are two main approaches for generating tumor selectivity: transductional and non-transductional targeting. Transductional targeting involves modifying the specificity of viral coat protein, thus increasing entry into target cells while reducing entry to non-target cells. Non-transductional targeting involves altering the genome of the virus so it can only replicate in cancer cells. This can be done by either transcription targeting, where genes essential for viral replication are placed under the control of a tumor-specific promoter, or by attenuation, which involves introducing deletions into the viral genome that eliminate functions that are dispensable in cancer cells, but not in normal cells. There are also other, slightly more obscure methods.

Chen et al., (2001) used CV706, a prostate-specific adenovirus, in conjunction with radiotherapy on prostate cancer in mice. The combined treatment results in a synergistic increase in cell death, as well as a significant increase in viral burst size (the number of virus particles released from each cell lysis).

ONYX-015 has undergone trials in conjunction with chemotherapy. The combined treatment gives a greater response than either treatment alone, but the results have not been entirely conclusive. ONYX-015 has shown promise in conjunction with radiotherapy.

Viral agents administered intravenously can be particularly effective against metastatic cancers, which are especially difficult to treat conventionally. However, bloodborne viruses can be deactivated by antibodies and cleared from the blood stream quickly e.g., by Kupffer cells (extremely active phagocytic cells in the liver, which are responsible for adenovirus clearance). Avoidance of the immune system until the tumour is destroyed could be the biggest obstacle to the success of oncolytic virus therapy. To date, no technique used to evade the immune system is entirely satisfactory. It is in conjunction with conventional cancer therapies that oncolytic viruses show the most promise, since combined therapies operate synergistically with no apparent negative effects.

The specificity and flexibility of oncolytic viruses means they have the potential to treat a wide range of cancers including lung cancer with minimal side effects. Oncolytic viruses have the potential to solve the problem of selectively killing cancer cells.

### Nanotherapy

Nanometer-sized particles have novel optical, electronic, and structural properties that are not available from either individual molecules or bulk solids. When linked with tumor-targeting moieties, such as tumor-specific ligands or monoclonal antibodies, these nanoparticles can be used to target cancer-specific receptors, tumor antigens (biomarkers), and tumor vasculatures with high affinity and precision. The formulation and manufacturing process for cancer nanotherapy is disclosed in patent U.S. Pat. No. 7,179,484, and article M. N. Khalid, P. Simard, D. Hoarau, A. Dragomir, J. Leroux, Long Circulating Poly(Ethylene Glycol)Decorated Lipid Nanocapsules Deliver Docetaxel to Solid Tumors, Pharmaceutical Research, 23(4), 2006, all of which are herein incorporated by reference in their entireties.

### RNA Therapy

RNA including but not limited to siRNA, shRNA, microRNA may be used to modulate gene expression and treat cancers. Double stranded oligonucleotides are formed by the assembly of two distinct oligonucleotide sequences where the oligonucleotide sequence of one strand is complementary to the oligonucleotide sequence of the second strand; such double stranded oligonucleotides are generally assembled from two separate oligonucleotides (e.g., siRNA), or from a single molecule that folds on itself to form a double stranded structure (e.g., shRNA or short hairpin RNA). These double stranded oligonucleotides known in the art all have a common feature in that each strand of the duplex has a distinct nucleotide sequence, wherein only one nucleotide sequence region (guide sequence or the antisense sequence) has complementarity to a target nucleic acid sequence and the other strand (sense sequence) comprises nucleotide sequence that is homologous to the target nucleic acid sequence.

MicroRNAs (miRNA) are single-stranded RNA molecules of about 21-23 nucleotides in length, which regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression.

Certain RNA inhibiting agents may be utilized to inhibit the expression or translation of messenger RNA (“mRNA”) that is associated with a cancer phenotype. Examples of such agents suitable for use herein include, but are not limited to, short interfering RNA (“siRNA”), ribozymes, and antisense oligonucleotides. Specific examples of RNA inhibiting agents suitable for use herein include, but are not limited to, Candy, Sirna-027, fomivirsen, and angiozyme.

### Small Molecule Enzymatic Inhibitors

Certain small molecule therapeutic agents are able to target the tyrosine kinase enzymatic activity or downstream signal transduction signals of certain cell receptors such as epidermal growth factor receptor (“EGFR”) or vascular endothelial growth factor receptor (“VEGFR”). Such targeting by small molecule therapeutics can result in anti-cancer effects. Examples of such agents suitable for use herein include, but are not limited to, imatinib, gefitinib, erlotinib, lapatinib, canertinib, ZD6474, sorafenib (BAY 43-9006), ERB-569, and their analogues and derivatives, as well as additional growth factor inhibitors disclosed herein.

### Anti-Metastatic Agents

The process whereby cancer cells spread from the site of the original tumor to other locations around the body is termed cancer metastasis. Certain agents have anti-metastatic properties, designed to inhibit the spread of cancer cells. Examples of such agents suitable for use herein include, but are not limited to, marimastat, bevacizumab, trastuzumab, rituximab, erlotinib, MMI-166, GRN163L, hunter-killer peptides, tissue inhibitors of metalloproteinases (TIMPs), their analogues, derivatives and variants.

### Chemopreventative Agents

Certain chemopreventative agents may be used in combination with any of the methods provided herein. These agents can be used to prevent initial occurrences of cancer, or to prevent recurrence or metastasis. Administration with such chemopreventative agents in combination with eflornithine-NSAID conjugates of the invention can act to both treat and prevent the recurrence of cancer. Examples of chemopreventative agents suitable for use herein include, but are not limited to, tamoxifen, raloxifene, tibolone, bisphosphonate, ibandronate, estrogen receptor modulators, aromatase inhibitors (letrozole, anastrozole), luteinizing hormone-releasing hormone agonists, goserelin, vitamin A, retinal, retinoic acid, fenretinide, 9-cis-retinoid acid, 13-cis-retinoid acid, all-trans-retinoic acid, isotretinoin, tretinoid, vitamin B6, vitamin B12, vitamin C, vitamin D, vitamin E, cyclooxygenase inhibitors, non-steroidal anti-inflammatory drugs (NSAIDs), aspirin, ibuprofen, celecoxib, polyphenols, polyphenol E, green tea extract, folic acid, glucaric acid, interferon-alpha, anethole dithiolethione, zinc, pyridoxine, finasteride, doxazosin, selenium, indole-3-carbinal, alpha-difluoromethylornithine, carotenoids, beta-carotene, lycopene, antioxidants, coenzyme Q10, flavonoids, quercetin, curcumin, catechins, epigallocatechin gallate, N-acetylcysteine, indole-3-carbinol, inositol hexaphosphate, isoflavones, glucanic acid, rosemary, soy, saw palmetto, and calcium. An additional example of chemopreventative agents suitable for use in the present invention is cancer vaccines. These can be created through immunizing an individual with all or part of a cancer cell type that is targeted by the vaccination process.

### Dosing Regimen, Routes of Administration, and Formulations

In any of the methods of treating squamous cell lung cancer (e.g., stage IV squamous cell lung cancer), any one of the dosage or dosing schedule for (i) 4-iodo-3-nitrobenzamide, a metabolite thereof, or a pharmaceutically acceptable salt thereof; (ii) gemcitabine; and (iii) carboplatin described herein may be used.

The dosage of 4-iodo-3-nitrobenzamide, a metabolite thereof, or a pharmaceutically acceptable salt thereof may vary depending upon the individual's age, height, weight, overall health, etc. In some embodiments, the dosage of 4-iodo-3-nitrobenzamide (or a metabolite thereof, or a pharmaceutically acceptable salt thereof) is in the range of any one of about 0.1 mg/kg to about 50 mg/kg, about 1 mg/kg to about 50 mg/kg, about 1 mg/kg to about 100 mg/kg, about 1 mg/kg to about 25 mg/kg, about 2 mg/kg to about 50 mg/kg, about 2 mg/kg to about 40 mg/kg, about 3 mg/kg to about 30 mg/kg, about 4 mg/kg to about 20 mg/kg, or about 4 to about 15 mg/kg, about 2 mg/kg, about 4 mg/kg, about 5 mg/kg, about 5.6 mg/kg, about 6 mg/kg, about 8 mg/kg, about 10 mg/kg, about 11 mg/kg, about 11.2 mg/kg, about 12 mg/kg, about 15 mg/kg, about 20 mg/kg, about 25 mg/kg, about 30 mg/kg, about 35 mg/kg, about 40 mg/kg, about 50 mg/kg, about 60 mg/kg, about 75 mg/kg, or about 90 mg/kg. In some embodiments, 4-iodo-3-nitrobenzamide (or a metabolite thereof, or a pharmaceutically acceptable salt thereof) is administered at least any of about 2 mg/kg, about 4 mg/kg, about 5 mg/kg, about 5.6 mg/kg, about 6 mg/kg, about 8 mg/kg, about 10 mg/kg, about 11 mg/kg, about 11.2 mg/kg, about 12 mg/kg, about 15 mg/kg, about 20 mg/kg, about 25 mg/kg, about 30 mg/kg, about 35 mg/kg, about 40 mg/kg, about 50 mg/kg, about 60 mg/kg, about 75 mg/kg, or about 90 mg/kg. 4-iodo-3-nitrobenzamide (or a metabolite thereof, or a pharmaceutically acceptable salt thereof) may be administered intravenously, e.g., by IV infusion over about 10 to about 300 minutes, about 30 to about 180 minutes, about 45 to about 120 minutes or about 60 minutes (i.e., about 1 hour). In some embodiments, 4-iodo-3-nitrobenzamide (or a metabolite thereof, or a pharmaceutically acceptable salt thereof) may alternatively be administered orally. In this context, the term “about” has its normal meaning of approximately. In some embodiments, about means±10% or ±5%.

The dosage of gemcitabine may vary depending upon the individual's age, height, weight, overall health, etc. In some embodiments, the dosage of the gemcitabine is in the range of about 10 mg/m² to about 1000 mg/m², about 25 mg/m² to about 500 mg/m², 100 mg/m² to about 5000 mg/m², about 200 mg/m² to about 4000 mg/m², about 300 mg/m² to about 3000 mg/m², about 400 mg/m² to about 2000 mg/m², about 500 mg/m² to about 1500 mg/m², about 800 mg/m² to about 1500 mg/m², or about 800 mg/m² to about 1200 mg/m². In some embodiments, gemcitabine is administered at about any of 50 mg/m², 75 mg/m², 100 mg/m², 125 mg/m², 150 mg/m², 175 mg/m², 200 mg/m², 250 mg/m², 300 mg/m², 400 mg/m², 450 mg/m², 500 mg/m², 550 mg/m², 600 mg/m², 650 mg/m², 700 mg/m², 750 mg/m², 800 mg/m², 850 mg/m², 900 mg/m², 1000 mg/m², 1050 mg/m², 1100 mg/m², 1150 mg/m², 1200 mg/m², 1250 mg/m², 1300 mg/m², 1350 mg/m², 1400 mg/m², 1450 mg/m², 1500 mg/m², 1550 mg/m², 1600 mg/m², 1700 mg/m², 1800 mg/m², 1900 mg/m², or 2000 mg/m². Gemcitabine may be administered at least any of or at least about any of 50 mg/m², 75 mg/m², 100 mg/m², 125 mg/m², 150 mg/m², 175 mg/m², 200 mg/m², 250 mg/m², 300 mg/m², 400 mg/m², 450 mg/m², 500 mg/m², 550 mg/m², 600 mg/m², 650 mg/m², 700 mg/m², 750 mg/m², 800 mg/m², 850 mg/m², 900 mg/m², 1000 mg/m², 1050 mg/m², 1100 mg/m², 1150 mg/m², 1200 mg/m², 1250 mg/m², 1300 mg/m², 1350 mg/m², 1400 mg/m², 1450 mg/m², 1500 mg/m², 1550 mg/m², 1600 mg/m², 1700 mg/m², 1800 mg/m², 1900 mg/m², or 2000 mg/m². Gemcitabine may be administered intravenously, e.g., by IV infusion over about 10 to about 500 minutes, about 10 to about 300 minutes, about 30 to about 180 minutes, about 30 to about 60 minutes, about 45 to about 120 minutes, about 60 minutes (i.e. about 1 hour), or about 30 minutes. In some embodiments, gemcitabine may alternatively be administered orally.

Carboplatin may vary depending upon the individual's age, height, weight, overall health, etc. The dosage of carboplatin is determined by calculating the area under the blood plasma concentration versus time curve (AUC) in mg/mL·minute by methods known to those skilled in the cancer chemotherapy art, taking into account the individual's renal activity estimated by measuring creatinine clearance or glomerular filtration rate. In some embodiment, carboplatin is administered at any of about AUC 1 mg/ml·minute (“AUC 1”) to about AUC 8, about AUC 2 to about AUC 6, about AUC 3 to about AUC 6, or about AUC 4 to about AUC 6. In some embodiments, carboplatin is administered at any of about 0.1 to about 6 mg/ml·min, about 1 to about 3 mg/ml·min, about 1.5 to about 2.5 mg/ml·min, about 1.75 to about 2.25 mg/ml·min or about 2 mg/ml·min. In some embodiments, carboplatin is administered at least about any of AUC 1, AUC 1.5, AUC 2, AUC 2.5, AUC 3, AUC 3.5, AUC 4, AUC 4.5, AUC 5, AUC 5.5, AUC 6, AUC 6.5, or AUC 7. In some embodiments, carboplatin is administered at about any of AUC 1, AUC 1.5, AUC 2, AUC 2.5, AUC 3, AUC 3.5, AUC 4, AUC 4.5, AUC 5, AUC 5.5, AUC 6, AUC 6.5, or AUC 7. Alternatively, the dosage of carboplatin is calculated based on the individual's body surface area. In some embodiments, a suitable dose of carboplatin is about 10 to about 500 mg/m², about 50 to about 400 mg/m², or about 50 to about 300 mg/m², e.g., about 50 mg/m², about 100 mg/m², about 200 mg/m², about 300 mg/m², about 350 mg/m², about 360 mg/m², about 400 mg/m², or about 450 mg/m². Carboplatin may be administered intravenously (IV), for example, carboplatin may be administered intravenously (IV) over a period of about 10 to about 500 minutes, about 30 to about 400 minutes, about 60 to about 300 minutes, about 100 to about 250 minutes, about 60 minutes, about 120 minutes, about 150 minutes, about 180 minutes, about 210 minutes, about 240 minutes, about 270 minutes, about 300 minutes, about 330 minutes, or about 360 minutes. In this context, the term “about” has its normal meaning of approximately. In some embodiments, about means±10% or ±5%.

In some embodiments, the method of treating squamous non-small cell lung cancer (“squamous cell lung cancer”) (e.g., stage IV squamous cell lung cancer) in an individual comprises at least one treatment cycle comprising administering to the individual having squamous cell lung cancer an effective amount of: (i) 4-iodo-3-nitrobenzamide, a metabolite thereof, or a pharmaceutically acceptable salt thereof; (ii) gemcitabine; and (iii) carboplatin. For example, the method may comprise at least 2 (e.g., about any of or at least about any of 2, 3, 4, 5, 6, 7, 8, 9, or 10) treatment cycles. In some embodiments, the treatment includes at most any of 2 cycles, 3 cycles, 4 cycles, 5 cycles, 6 cycles, 7 cycles, 8 cycles, 9 cycles, or 10 cycles. In some embodiments, the treatment comprises a treatment cycle of at least about any of 1 week, 10 days, 2 weeks, 3 weeks, 4 weeks, 5 weeks, 6 weeks, 7 weeks, 8 weeks, 9 weeks, 10 weeks, 12 weeks, or 15 weeks. A treatment cycle may be a period of about any of 1 week, 10 days, 2 weeks, 3 weeks, 4 weeks, 5 weeks, 6 weeks, 7 weeks, 8 weeks, 9 weeks, or 10 weeks.

Administration of (i) 4-iodo-3-nitrobenzamide, a metabolite thereof, or a pharmaceutically acceptable salt thereof; (ii) gemcitabine; and (iii) carboplatin may be sequential, separate, or simultaneous. Administration of (i) 4-iodo-3-nitrobenzamide, a metabolite thereof, or a pharmaceutically acceptable salt thereof; (ii) gemcitabine; and (iii) carboplatin may be on different days of a treatment cycle, such as the treatment cycles described herein. The interval between administration of 4-iodo-3-nitrobenzamide, gemcitabine, and carboplatin may vary within a treatment cycle (e.g., administration is not always spaced apart by 7 day, but may be at intervals of 1 day followed by an interval of 9 days, etc.). Similarly, at certain times during the treatment cycle, 4-iodo-3-nitrobenzamide, gemcitabine, and carboplatin may be administered at the same time, and at other points during the treatment administered at different times.

4-iodo-3-nitrobenzamide (or a metabolite thereof, or a pharmaceutically acceptable salt thereof) may be administered every day of the treatment cycle, or administered on certain days but not on every day of the treatment cycle. In some embodiments, 4-iodo-3-nitrobenzamide (or a metabolite thereof, or a pharmaceutically acceptable salt thereof) is administered daily, once a week, once every three weeks, twice a week, twice every three weeks, three times a week, four times a week, four times every three weeks, five times a week, six times a week, once 10 days, once two weeks, once four weeks, once six weeks, or once eight weeks. 4-iodo-3-nitrobenzamide (or a metabolite thereof, or a pharmaceutically acceptable salt thereof) may be administered on the selected days of each treatment cycle, for example, 4-iodo-3-nitrobenzamide is administered daily on 2 (or 3, 4, 5, 6, 7, 8, 9, 10) days of the treatment cycle, and 4-iodo-3-nitrobenzamide is not administered on other days of the treatment cycle. 4-iodo-3-nitrobenzamide (or a metabolite thereof, or a pharmaceutically acceptable salt thereof) may be administered (e.g., at about 5.6 mg/kg) on 4 days of a treatment cycle, e.g., on days 1, 4, 8, 11 of a 21-day treatment cycle. 4-iodo-3-nitrobenzamide (or a metabolite thereof, or a pharmaceutically acceptable salt thereof) may be administered (e.g., at about 11.2 mg/kg) on 2 days of a treatment cycle, e.g., on days 1 and 8 of a 21-day treatment cycle.

In some embodiments, the treatment comprises a treatment cycle of at least 11 days, wherein on days 1, 4, 8 and 11 of the cycle, the individual receives about 10 to about 100 mg/kg of 4-iodo-3-nitrobenzamide or a molar equivalent of a metabolite thereof. In some embodiments, the treatment comprises a treatment cycle of at least 11 days, wherein on days 4, 8 and 11 of the cycle, the individual receives about 1 to about 50 mg/kg of 4-iodo-3-nitrobenzamide or a molar equivalent of a metabolite thereof. In some embodiments, the treatment comprises a treatment cycle of at least 11 days, wherein on days 1, 4, 8 and 11 of the cycle, the individual receives about 1, 2, 3, 4, 5, 5.6, 6, 8, 9, 10, 11, 11.2, 12, 14, 16, 18, or 20 mg/kg of 4-iodo-3-nitrobenzamide. In some embodiments, the treatment cycle is about any of 2 weeks, 3 weeks, or 4 weeks.

Gemcitabine may be administered daily, e.g., every day of the treatment cycle, or administered on certain days but not on every day of the treatment cycle. In some embodiments, gemcitabine is administered daily, once a week, twice a week, twice every 3 weeks, three times a week, four times a week, five times a week, six times a week, once every 10 days, once every two weeks, once every three weeks, once every four weeks, once every six weeks, or once every eight weeks. Gemcitabine may be administered on the selected days of each treatment cycle, for example, gemcitabine is administered daily on 2 (or 3, 4, 5, 6, 7, 8, 9, 10) days of the treatment cycle, and gemcitabine is not administered on other days of the treatment cycle. Gemcitabine may be administered (e.g., at about 1000 mg/m²) on 2 days of a treatment cycle, e.g., on days 1 and 8 of a 21-day treatment cycle. In some embodiments, the treatment cycle is about any of 2 weeks, 3 weeks, or 4 weeks.

Carboplatin may be administered daily, e.g., every day of the treatment cycle, or administered on certain days but not on every day of the treatment cycle. In some embodiments, carboplatin is administered daily, once a week, twice a week, twice every 3 weeks, three times a week, four times a week, five times a week, six times a week, once every 10 days, once every two weeks, once every three weeks, once every four weeks, once every six weeks, or once every eight weeks. Carboplatin may be administered on the selected days of each treatment cycle, for example, carboplatin is administered daily on 1 (or 2, 3, 4, 5, 6, 7, 8, 9, 10) day(s) of the treatment cycle, and carboplatin is not administered on other days of the treatment cycle. Carboplatin may be administered (e.g., at about AUC 5) on 1 day of a treatment cycle, e.g., on day 1 of a 21-day treatment cycle. In some embodiments, the treatment cycle is about any of 2 weeks, 3 weeks, or 4 weeks.

In some embodiments, there is provided a method of treating squamous cell lung cancer (e.g., stage IV squamous cell lung cancer) in an individual, comprising administering to the individual having squamous cell lung cancer an effective amount of: (i) 4-iodo-3-nitrobenzamide, a metabolite thereof, or a pharmaceutically acceptable salt thereof; (ii) gemcitabine; and (iii) carboplatin, wherein (i) carboplatin is administered to the individual at about AUC 3 to about AUC 6; (ii) gemcitabine is administered to the individual at about 800 mg/m² to about 1500 mg/m²; and (iii) 4-iodo-3-nitrobenzamide or a metabolite thereof or a pharmaceutically acceptable salt thereof is administered to the individual at about 4 mg/kg to about 15 mg/kg. In some embodiments, 4-iodo-3-nitrobenzamide is administered.

In some embodiments of any one of the methods provided herein, the method comprises at least one treatment cycle of 21-days comprising administering (i) 4-iodo-3-nitrobenzamide, a metabolite thereof, or a pharmaceutically acceptable salt thereof; (ii) gemcitabine; and (iii) carboplatin to an individual, wherein (i) carboplatin is administered to the individual at about 5 mg/ml·minute (AUC 5) on day 1 of the treatment cycle; (ii) gemcitabine is administered to the individual at a dose of about 1000 mg/m² on days 1 and 8 of the treatment cycle; and (iii) 4-iodo-3-nitrobenzamide or a metabolite thereof or a pharmaceutically acceptable salt thereof is administered to the individual at a dose of about 5.6 mg/kg twice weekly on days 1, 4, 8, and 11 of the treatment cycle. In some embodiments, there is provided a method of treating squamous cell lung cancer (e.g., stage IV squamous cell lung cancer) in an individual, comprising administering to the individual having squamous cell lung cancer an effective amount of: (i) 4-iodo-3-nitrobenzamide, a metabolite thereof, or a pharmaceutically acceptable salt thereof; (ii) gemcitabine; and (iii) carboplatin, wherein the effective amount is administered over a 21-day treatment cycle, wherein (i) carboplatin is administered to the individual at about 5 mg/ml·minute (AUC 5) on day 1 of the treatment cycle; (ii) gemcitabine is administered to the individual at about 1000 mg/m² on days 1 and 8 of the treatment cycle; and (iii) 4-iodo-3-nitrobenzamide or a metabolite thereof or a pharmaceutically acceptable salt thereof is administered to the individual at about 5.6 mg/kg on days 1, 4, 8, and 11 of the treatment cycle. In some embodiments, the individual receives at least 2 treatment cycles (e.g., about any of or at least about any of 2, 3, 4, 5, 6, 7, 8, 9, or 10 cycles).

In some cases, a beneficial effect is achieved when the administration of gemcitabine and carboplatin is temporally removed from the administration of the 4-iodo-3-nitrobenazmide (or pharmaceutically acceptable salt or solvate thereof, or metabolite thereof) by a significant period of time (e.g., about 12 hours, about 24 hours, about 36 hours, about 48 hours, etc.), or, for example, when administration is spaced apart by at least 1 day, 2 days, 3 days, 4 days, 5 days, 6 days, 7 days, 8 days, 9 days, 10 days, etc.).

4-iodo-3-nitrobenzamide (or a metabolite thereof or a pharmaceutically acceptable salt thereof), gemcitabine and carboplatin may be co-administered to the individual. Co-administration is meant to include simultaneous or sequential administration of the compounds individually or in combination (more than one compound), such as described herein.

4-iodo-3-nitrobenzamide (or a metabolite thereof or a pharmaceutically acceptable salt thereof), gemcitabine and carboplatin may be continuously or not continuously given to an individual. “Not continuously” means that the compound or composition provided herein is not administered to the individual over a period of time, e.g., there is a resting period when the individual does not receive the compound or composition. It may be that one compound is administered continuously administered to an individual while the second compound is not administered continuously administered to the individual.

4-iodo-3-nitrobenzamide (or a metabolite thereof or a pharmaceutically acceptable salt thereof), gemcitabine and/or carboplatin may be formulated in separate formulations or in the same formulation. 4-iodo-3-nitrobenzamide (or a metabolite thereof or a pharmaceutically acceptable salt thereof), gemcitabine and carboplatin may be administered through different administration route or using same administration routes. In some embodiments, there is provided a composition (e.g., pharmaceutical composition) comprising 4-iodo-3-nitrobenzamide (or a metabolite thereof or a pharmaceutically acceptable salt thereof), gemcitabine and/or carboplatin.

In some embodiments, there are provided formulations (e.g., pharmaceutical formulations) comprising 4-iodo-3-nitrobenzamide (or a metabolite thereof, or a pharmaceutically acceptable salt thereof), gemcitabine and/or carboplatin, and/or a carrier, such as a pharmaceutically acceptable carrier. The formulations may include optical isomers, diastereomers, carriers of the compounds disclosed herein. In some embodiments, the carrier is a cyclodextrin, or a derivative thereof, e.g., hydroxypropyl-β-cyclodextrin (HPBCD). In some embodiments, the formulations are formulated for intravenous administration.

A formulation may comprise both the 4-iodo-3-nitrobenzamide compound and acid forms in particular proportions, depending on the relative potencies of each and the intended indication. The two forms may be formulated together or in different formulations. They may be in the same dosage unit e.g. in one cream, suppository, tablet, capsule, or packet of powder to be dissolved in a beverage; or each form may be formulated in a separate unit, e.g., two creams, two suppositories, two tablets, two capsules, a tablet and a liquid for dissolving the tablet, a packet of powder and a liquid for dissolving the powder, etc.

The pharmaceutical compositions of the present invention may be provided as a prodrug and/or may be allowed to interconvert to 4-iodo-3-nitrobenzamide form in vivo after administration. That is, either 4-iodo-3-nitrobenzamide or metabolites thereof or pharmaceutically acceptable salts may be used in developing a formulation for use in the present invention.

The pharmaceutical compositions of 4-iodo-3-nitrobenzamide (or a metabolite thereof or a pharmaceutically acceptable salt thereof), gemcitabine and carboplatin can be combined with other active ingredients, such as other chemotherapeutic agents as described herein. The three compounds may be formulated together, in the same dosage unit e.g., in one cream, suppository, tablet, capsule, or packet of powder to be dissolved in a beverage; or each form may be formulated in separate units, e.g., three creams, three suppositories, three tablets, three capsules, a tablet and a liquid for dissolving the tablet, a packet of powder and a liquid for dissolving the powder, etc.

The term “pharmaceutically acceptable salt” means those salts which retain the biological effectiveness and properties of the compounds used in the present invention, and which are not biologically or otherwise undesirable. For example, a pharmaceutically acceptable salt does not interfere with the beneficial effect of the compound of the invention in treating non-small cell lung cancer (e.g., squamous cell lung cancer).

Typical salts are those of the inorganic ions, such as, for example, sodium, potassium, calcium and magnesium ions. Such salts include salts with inorganic or organic acids, such as hydrochloric acid, hydrobromic acid, phosphoric acid, nitric acid, sulfuric acid, methanesulfonic acid, p toluenesulfonic acid, acetic acid, fumaric acid, succinic acid, lactic acid, mandelic acid, malic acid, citric acid, tartaric acid or maleic acid. In addition, where compounds contain a carboxy group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine.

For injection, the 4-iodo-3-nitrobenzamide or pharmaceutically acceptable salt thereof may be formulated for administration in aqueous solutions, preferably in physiologically compatible buffers such as phosphate buffers, Hank's solution, or Ringer's solution. Such compositions may also include one or more excipients, for example, preservatives, solubilizers, fillers, lubricants, stabilizers, albumin, and the like. Formulations of 4-iodo-3-nitrobenzamide are described in US Pat. Publ. No. 2008/0176946 A1, which is incorporated by reference in its entirety, particularly with reference to intravenous (e.g., hydroxypropyl-(β-cyclodextrin, etc.) and oral (e.g., sodium lauryl sulfate, etc.) formulations. In some embodiments, 4-iodo-3-nitrobenzamide is formulated in 25% (w/v) hydroxypropyl-β-cyclodextrin and 10 mM phosphate buffer for intravenous administration as described in U.S. patent application Ser. No. 12/510,969, filed Jul. 28, 2009, with U.S. Publication No. 2010/0160442, which is incorporated herein by reference.

Additional methods of formulation, such as for gemcitabine and carboplatin, are known in the art, for example, as disclosed in Remington's Pharmaceutical Sciences, latest edition, Mack Publishing Co., Easton, Pa. Compositions described herein may also be formulated for transmucosal administration, buccal administration, for administration by inhalation, for parental administration, for transdermal administration, and rectal administration.

Pharmaceutical compositions suitable for use as described herein include compositions wherein the active ingredients are present in an effective amount, i.e., in an amount effective to achieve therapeutic and/or prophylactic benefit in squamous cell lung cancer described herein. The actual amount effective for a particular administration will depend on squamous cell lung cancer being treated, the condition of the individual, the formulation, and the route of administration, as well as other factors known to those of skill in the art in view of the specific teaching provided herein. In light of the disclosure herein, optimization of an effective amount of 4-iodo-3-nitrobenzamide, gemcitabine, and/or carboplatin provided herein, within the ranges specified, may be determined.

In some embodiments, the composition is administered in unit dosage form. In some embodiments, the unit dosage form is adapted for oral or parenteral administration. In some embodiments, upon administration of the composition, at least one therapeutic effect is obtained, said at least one therapeutic effect being reduction in size of a tumor, reduction in metastasis, complete remission, partial remission, pathologic complete response, increase in overall response rate, or stable disease.

The compositions described herein may be administered to an individual through appropriate route, such as, but are not limited to intradermal, intramuscular, intraperitoneal, intravenous, intraarterial, subcutaneous, intranasal, epidural, and oral routes. In some embodiments, the composition or compound(s) provided herein is administered by the parenteral route, e.g., intravenously, intraperitoneally, subcutaneously, intradermally, or intramuscularly. Compositions may be administered by any convenient route, for example by infusion or bolus injection, by absorption through epithelial or mucocutaneous linings (e.g., oral mucosa, rectal and intestinal mucosa, etc.) and may be administered in combination with other biologically active agents, e.g., such as described herein. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir.

### Kits

Also provided are kits for administration of 4-iodo-3-nitrobenzamide or a metabolite thereof or a pharmaceutically acceptable salt thereof, gemcitabine and carboplatin as provided herein. The kits may include an instruction for using 4-iodo-3-nitrobenzamide or a metabolite thereof or a pharmaceutically acceptable salt thereof, gemcitabine and carboplatin to treat squamous cell lung cancer (e.g., stage IV squamous cell lung cancer). The instruction may be in accordance with any one of the methods provided herein. The instructions may be in any suitable format, including, but not limited to, printed matter, videotape, computer readable disk, optical disc or directions to internet-based instructions.

In certain embodiments, the kits may include a dosage amount of at least one composition as disclosed herein. Kits may further comprise suitable packaging and/or instructions for use of the formulation. Kits may also comprise a means for the delivery of the formulation thereof.

In some embodiments, there is provided a kit comprising 4-iodo-3-nitrobenzamide, a metabolite thereof, or a pharmaceutically acceptable salt thereof. The kit may further comprise instructions for using 4-iodo-3-nitrobenzamide, a metabolite thereof, or a pharmaceutically acceptable salt thereof in combination with gemcitabine and carboplatin for treatment of squamous cell lung cancer in an individual. In some embodiments, the instructions comprise administering an effective amount of (i) 4-iodo-3-nitrobenzamide, a metabolite thereof, or a pharmaceutically acceptable salt thereof, (ii) gemcitabine, and (iii) carboplatin over a 21-day treatment cycle to the individual, wherein (i) carboplatin is administered to the individual at about 5 mg/ml·minute (AUC 5) on day 1 of the treatment cycle; (ii) gemcitabine is administered to the individual at about 1000 mg/m² on days 1 and 8 of the treatment cycle; and (iii) 4-iodo-3-nitrobenzamide or a metabolite thereof or a pharmaceutically acceptable salt thereof is administered to the individual at about 5.6 mg/kg on days 1, 4, 8, and 11 of the treatment cycle. In some embodiments, the squamous cell lung cancer is stage IV. In some embodiments, the instructions are included in a package insert or a label.

In some embodiments, there is provided a kit comprising (i) 4-iodo-3-nitrobenzamide, a metabolite thereof, or a pharmaceutically acceptable salt thereof, (ii) gemcitabine, and/or (iii) carboplatin. The kit may further comprise instructions for using (i) 4-iodo-3-nitrobenzamide, a metabolite thereof, or a pharmaceutically acceptable salt thereof; (ii) gemcitabine; and (iii) carboplatin for treatment of squamous cell lung cancer in an individual. In some embodiments, the instructions comprise administering an effective amount of (i) 4-iodo-3-nitrobenzamide, a metabolite thereof, or a pharmaceutically acceptable salt thereof, (ii) gemcitabine, and (iii) carboplatin over a 21-day treatment cycle to the individual, wherein (i) carboplatin is administered to the individual at about 5 mg/ml·minute (AUC 5) on day 1 of the treatment cycle; (ii) gemcitabine is administered to the individual at about 1000 mg/m² on days 1 and 8 of the treatment cycle; and (iii) 4-iodo-3-nitrobenzamide or a metabolite thereof or a pharmaceutically acceptable salt thereof is administered to the individual at about 5.6 mg/kg on days 1, 4, 8, and 11 of the treatment cycle. In some embodiments, the squamous cell lung cancer is stage IV. In some embodiments, the instructions are included in a package insert or a label.

The kits may include other pharmaceutical agents (such as the side-effect limiting agents, chemotherapy agents, gene therapy agents, DNA therapy agents, RNA therapy agents, viral therapy agents, nanotherapy agents, small molecule enzymatic inhibitors, anti-metastatic agents, etc.), for use in conjunction with 4-iodo-3-nitrobenzamide or a metabolite thereof or a pharmaceutically acceptable salt thereof, gemcitabine, and carboplatin. These agents may be provided in a separate form, or mixed with 4-iodo-3-nitrobenzamide or a metabolite thereof or a pharmaceutically acceptable salt thereof, gemcitabine, and carboplatin, provided such mixing does not reduce the effectiveness of 4-iodo-3-nitrobenzamide (or a metabolite thereof or a pharmaceutically acceptable salt thereof), gemcitabine, or carboplatin, and is compatible with the route of administration. Similarly, the kits may include additional agents for adjunctive therapy or other agents known to the skilled artisan as effective in the treatment or prevention of squamous cell lung cancer described herein.

The kits may optionally include appropriate instructions for preparation and administration of the composition, side effects of the composition, and any other relevant information.

In another aspect, provided are kits for treating an individual who suffers from or is susceptible to the squamous cell lung cancer (e.g., stage IV squamous cell lung cancer) described herein, comprising a first container comprising a dosage amount of a formulation as disclosed herein, and instructions for use. The container may be any of those known in the art and appropriate for storage and delivery of intravenous formulation. In certain embodiments, the kit further comprises a second container comprising a pharmaceutically acceptable carrier, diluent, adjuvant, etc. for preparation of the composition to be administered to the individual. In some embodiments, the kit further comprises a second container comprising a different compound than the compound contained in the first container. For example, the first container may comprise a composition comprising 4-iodo-3-nitrobenzamide or a metabolite thereof or a pharmaceutically acceptable salt thereof and the second container may comprise gemcitabine and/or carboplatin

Kits may also be provided that contain sufficient dosages to provide effective treatment for an individual for an extended period, such as 1-3 days, 1-5 days, a week, 2 weeks, 3, weeks, 4 weeks, 6 weeks, 8 weeks, 3 months, 4 months, 5 months, 6 months, 7 months, 8 months, 9 months or more.

Kits may also include multiple doses of the compounds and instructions for use and packaged in quantities sufficient for storage and use in pharmacies, for example, hospital pharmacies and compounding pharmacies.

The kits may include the compounds as described herein packaged in either a unit dosage form or in a multi-use form. The kits may also include multiple units of the unit dose form. In certain embodiments, provided are the compound described herein in a unit dose form. In other embodiments, the compositions may be provided in a multi-dose form (e.g., a blister pack, etc.).

Also provided are articles of manufacture comprising the compositions described herein in suitable packaging. Suitable packaging for compositions described herein are known in the art, and include, for example, vials (such as sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed.

The examples below are intended to be purely exemplary of the invention and should therefore not be considered to limit the invention in any way. The following examples and detailed description are offered by way of illustration and not by way of limitation.

### EXAMPLES

### Example 1

### Study of administering 4-iodo-3-nitrobenzamide, gemcitabine, and carboplatin in squamous cell lung cancer

A study to assess the efficacy of 4-iodo-3-nitrobenzamide administered in combination with gemcitabine and carboplatin for the treatment of squamous cell lung cancer, a non-small cell lung cancer, is conducted.

Primary Endpoint:

To assess overall survival (OS) in patients with advanced squamous cell lung cancer receiving 4-iodo-3-nitrobenzamide (BA) administered as a 60-minute intravenous infusion twice weekly in combination with a gemcitabine/carboplatin chemotherapy regimen (GEM/CARBO).

Secondary Endpoints:

(1) To assess the safety profiles of the treatment described in this Example; (2) To assess the progression free survival (PFS), overall response rate (ORR), and time to progression (TTP) in both arms; (3) To assess the relationship between DNA repair pathway characteristics of tumors at baseline and clinical outcome of disease; and (4) To assess patient quality of life.

The duration of the study for a patient includes a period for inclusion of up to 3 weeks. The patients may continue treatment up to a maximum of 6 cycles or until disease progression, unacceptable toxicity or consent withdrawal, followed by a minimum of 30-day follow-up after the last study treatment administration. Patients are followed for at least 30 days after the last administration of study treatment for safety purposes. In case of study treatment discontinuation without disease progression, efficacy data is collected every 6 weeks until disease progression, death or end of study whatever comes first. After disease progression, the patient is followed up every 12 weeks (3 months) for overall survival (OS) until death or end of study whatever comes first. The end of the study is one year after the first dose of the last treated patient.

The experimental arm of this clinical trial receives 4-iodo-3-nitrobenzamide (“BA”), gemcitabine, and carboplatin. 4-iodo-3-nitrobenzamide is administered at a dose of 5.6 mg/kg as a 60-minute intravenous (“IV”) infusion on days 1, 4, 8, and 11 of a 21-day treatment cycle. The 4-iodo-3-nitrobenzamide infusion starts after administration of the gemcitabine/carboplatin regimen. Gemcitabine is administered at 1000 mg/m² as a 30-minute IV infusion on days 1 and 8 of a 21-day treatment cycle, and carboplatin is administered at AUC 5 (5 mg/ml·minute) as a 3- to 4-hour IV infusion on day 1 of a 21-day treatment cycle after the end of gemcitabine infusion.

The control arm of this clinical trial receives gemcitabine, and carboplatin. Gemcitabine is administered at 1000 mg/m² as a 30-minute IV infusion on days 1 and 8 of a 21-day treatment cycle, and carboplatin is administered at AUC 5 (5 mg/ml·minute) as a 3- to 4-hour IV infusion on day 1 of a 21-day treatment cycle after the end of gemcitabine infusion. Patients are restaged by CT scan according to criteria set forth in the Response Evaluation Criteria in Solid Tumors, version 1.1 (RECIST 1.1) every two cycles/six weeks. Patients can remain on study after six cycles/eighteen weeks if there is no evidence of progressive disease or the presence of dose-limiting toxicities. Finally, crossover from the gemcitabine/carboplatin arm to the 4-iodo-3-nitrobenzamide/gemcitabine/carboplatin arm is allowed upon disease progression.

The primary endpoint is overall survival (OS). The secondary endpoints are progression free survival (PFS), overall response rate (ORR), and time to progression (TTP) in both arms, as well as an assessment of treatment safety/tolerability and patient quality of life.

Eligible patients must meet the following inclusion criteria to be enrolled in the study: (1) Newly diagnosed, stage IV squamous cell lung cancer. This includes patients who present with disseminated metastases, and those with a malignant pleural or pericardial effusion (i.e., formerly stage IIIB in the 6th TNM staging system); (2) Patients who have received prior adjuvant therapy for early-stage lung cancer are eligible if at least 12 months have elapsed from that treatment; (3) Histologically confirmed squamous cell bronchogenic carcinoma. Patients whose tumors contain mixed non-small cell histologies are eligible, as long as squamous carcinoma is the predominant histology. Mixed tumors with small cell anaplastic elements are not eligible. Cytologic specimens obtained by brushings, washings, or needle aspiration of the defined lesion are acceptable; (4) Patients with previous radiotherapy as definitive therapy for locally advanced non-small cell lung cancer are eligible, as long as the recurrence is outside the original radiation therapy port. Radiation therapy must have been completed >4 weeks prior to the date the Informed Consent is signed; (5) Presence of evaluable (measureable or non-measurable) disease; (6) ECOG Performance Status of 0 or 1; (7) Laboratory values as follows: (a) Absolute neutrophil count (ANC)>1,500/microL and platelets >100,000/microL (≦72 hours prior to initial treatment); (b) Hemoglobin >9 g/dL (Note: Patients may be transfused or receive erythropoietin to maintain or exceed this level); (c) Bilirubin <ULN; (d) Alanine aminotransferase (ALT) and aspartate aminotransferase (AST)≦2.5 times the upper limit of normal if no liver involvement or ≦5 times the upper limit of normal with liver involvement; and (d) Creatinine <2.0 mg/dL, or creatinine clearance >40 mL/min (as calculated by the Cockcroft-Gault method (see Section 8.3.3); (8) Women of childbearing potential must have a negative serum pregnancy test performed within 7 days prior to start of treatment. Women of childbearing potential or men with partners of childbearing potential must use effective birth control measures during treatment. If a woman becomes pregnant or suspects she is pregnant while participating in this study, she must agree to inform her treating physician immediately; (9) Patients must be over 18 years of age; (10) Ability to understand the nature of this study, give written informed consent, and comply with study requirements; and (11) Patients entering this study must be willing to provide tissue from a previous tumor biopsy (if available) for correlative testing. If tissue is not available, a patient would still be eligible for enrollment into the study.

Exclusion Criteria: (1) Prior treatment with gemcitabine, carboplatin (except in the adjuvant setting), or 4-iodo-3-nitrobenzamide; (2) Past or current history of neoplasm other than the entry diagnosis, with the exception of treated non-melanoma skin cancer or carcinoma in-situ of the cervix, or other cancers cured by local therapy alone and a disease-free survival of >5 years; (3) A history of cardiac disease, as defined by: (a) malignant hypertension; (b) unstable angina; (c) congestive heart failure; (d) myocardial infarction within the previous 6 months; and (e) symptomatic cardiac arrhythmias; (4) Active brain metastases (patients with treated brain metastases are eligible, if (a) radiation therapy was completed at least 2 weeks prior to study entry; (b) follow-up scan shows no disease progression; and (c) patient does not require steroids); (5) Women who are pregnant or lactating; (6) Any serious, active infection (>Grade 2) at the time of treatment; (7) A serious underlying medical condition that would impair the ability of the patient to receive protocol treatment; (8) A major surgical procedure, open biopsy, or significant traumatic injury ≦28 days of beginning treatment, or anticipation of the need for major surgery during the course of the study; (9) Uncontrolled or intercurrent illness including, but not limited to, ongoing or active infection, symptomatic congestive heart failure, unstable angina pectoris, or cardiac arrhythmia; (10) History of any medical or psychiatric condition or laboratory abnormality that, in the opinion of the investigator, may increase the risks associated with the study participation or administration of the investigational products, or that may interfere with the interpretation of the results; and (11) Known or suspected allergy/hypersensitivity to any agent given in the course of this trial. The above information is not intended to contain all considerations relevant to a patient's potential participation in a clinical trial.

### Example 2

### Randomized Phase III Trial of Gemcitabine/Carboplatin +/−4-iodo-3-nitrobenzamide in Patients with Previously Untreated Stage IV Squamous Non-Small Cell Lung Cancer (NSCLC)

Trial Design: Patients are randomized 1:1 to Arm A (with 4-iodo-3-nitrobenzamide) or Arm B (without 4-iodo-3-nitrobenzamide). All patients receive gemcitabine 1000 mg/m² intravenously (“IV”) on days 1 and 8, and carboplatin AUC=5 IV on day 1 of each 21-day cycle. Arm A patients also receive 4-iodo-3-nitrobenzamide 5.6 mg/kg IV on days 1, 4, 8, and 11. All patients are assessed for response per RECIST version 1.1 every 6 weeks. While 6 treatment cycles are planned, patients without evidence of progression or other reason for discontinuation may remain on treatment beyond 6 cycles. Accrual of 825 patients provides 90% power to detect an improvement in survival from 8 months (anticipated with gemcitabine/carboplatin) to 10.7 months.

Eligibility: Patients with newly diagnosed stage IV squamous NSCLC with ECOG PS 0-1 are eligible, including those with disseminated metastases or malignant pleural or pericardial effusions (stage IIIB in the 6th edition AJCC staging system). Patients may have had carboplatin in the adjuvant setting for early stage disease, but prior treatment with gemcitabine or 4-iodo-3-nitrobenzamide (iniparib) is not allowed. Additional exclusion criteria include: history of recent cardiac disease, untreated brain metastases, and treatment for early-stage lung cancer within 12 months of study entry.

### Example 3

### Randomized Phase III Trial of Gemcitabine/Carboplatin with or without 4-iodo-3-nitrobenzamide in Patients with Previously Untreated Stage IV Squamous Non-Small Cell Lung Cancer (NSCLC)

This randomized phase III trial is designed to evaluate the overall survival (OS) of patients with stage IV squamous lung cancer receiving gemcitabine/carboplatin with or without 4-iodo-3-nitrobenzamide.

Trial Design: This trial is a multi-center, randomized, open-label phase III study. Patients (N=825) are randomized 1:1 to Arm A (without 4-iodo-3-nitrobenzamide) or Arm B (with 4-iodo-3-nitrobenzamide). All patients receive gemcitabine 1000 mg/m² intravenously (“IV”) on days 1 and 8, and carboplatin AUC=5 IV on day 1 of each 21-day cycle. Arm B patients also receive 4-iodo-3-nitrobenzamide 5.6 mg/kg IV on days 1, 4, 8, and 11. Cycles are repeated every 21 days. All patients are assessed for response (RECIST 1.1) every 6 weeks (re-stage: by CT scans). Patients with disease progression are discontinued from the study. Six treatment cycles are planned, but patients without evidence of disease progression, or any other reason for discontinuation, may remain on treatment beyond 6 cycles. Follow-up of the patients are conducted.

The primary objective of the study is to evaluate the OS of patients with Stage IV squamous NSCLC receiving gemcitabine/carboplatin with or without 4-iodo-3-nitrobenzamide. The secondary objective of the study is to evaluate: (1) Progression-free survival; (2) Time to progression; (3) Objective response rate; (4) Safety and tolerability of the treatment regimen; and (5) Quality of life, as measured by EORTC QLQ-30 and QLQ-LC13 in patients with Stage IV squamous NSCLC receiving gemcitabine/carboplatin with or without 4-iodo-3-nitrobenzamide. The exploratory sub-study objective is to evaluate several biomarkers such as ERCC1 for possible correlation with clinical outcome.

Key eligibility criteria are described as follows. The inclusion criteria are: (1) Histologically confirmed squamous cell bronchogenic carcinoma (patients with mixed non-small histologies are eligible as long as squamous carcinoma is the predominant histology); (2) Stage IV at diagnosis or recurrent after resection±adjuvant chemotherapy for early stage disease (AJCC Staging System 7th edition); (3) No previous systemic treatment for advanced NSCLC; (4) ECOG performance status 0 or 1; (5) Willingness to provide tissue from a previous tissue biopsy (if available) for correlative testing (those with no tissue available are still eligible); (6) Adequate laboratory values as follows: (a) Absolute neutrophil count (ANC)≧1,500/μL and platelet count ≧100,000/μL (≦72 hours prior to initial treatment); (b) Hemoglobin >9 g/dL; (c) Bilirubin < the upper limit of normal (ULN); (d) Alanine aminotransferase (ALT) and aspartate aminotransferase (AST)≦2.5 times the upper limit of normal if no liver involvement, or ≦5 times the upper limit of normal with liver involvement; and (e) Creatinine ≦2.0 mg/dL, or creatinine clearance ≧40 mL/min (as calculated by the Cockcroft-Gault method); (7) Previous palliative radiation therapy allowed if completed ≧2 weeks prior to study entry. The exclusion criteria are: (1) Prior treatment with gemcitabine or 4-iodo-3-nitrobenzamide (no previous carboplatin except as part of adjuvant therapy); (2) Prior combined modality treatment (chemotherapy+radiation therapy) for locally advanced NSCLC; (3) Active brain metastases (patients with treated brain metastases are eligible if radiation therapy was completed at least 2 weeks before initiating study treatment, follow-up scans show no disease progression, and the patient does not require steroids); (4) A history of cardiac disease, defined as: (a) malignant hypertension; (b) unstable angina; (c) congestive heart failure; (d) myocardial infarction within 6 months; (e) symptomatic, unstable, or uncontrolled cardiac arrhythmias; and (f) patients who have stable, rate-controlled atrial fibrillation are eligible for study enrollment; (5) Major surgical procedure, open biopsy, or significant traumatic injury ≦28 days of beginning treatment; (6) any concurrent medical or psychiatric condition that increases the risks or affects the ability of the patient to participate in the study.

Statistical considerations: (1) Based on a two-sided log-rank test at the alpha=0.05 significance level, a sample of 750 patients (375 per Arm) provides a 90% power to detect a hazard ratio of 0.75; (2) The mean OS of the gemcitabine/carboplatin Arm is estimated at 8.0 months; (3) The addition of 4-iodo-3-nitrobenzamide to gemcitabine/carboplatin is anticipated to improve the median OS to 10.7 months; (4) Given the above parameters, 510 events are required; (5) To allow for an anticipated 10% drop-out rate, the study would enroll a total of 825 patients.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby

We claim:

1. A method of treating squamous cell lung cancer in an individual, comprising administering to the individual having squamous cell lung cancer an effective amount of: (i) 4-iodo-3-nitrobenzamide, a metabolite thereof, or a pharmaceutically acceptable salt thereof; (ii) gemcitabine; and (iii) carboplatin.

2. The method of claim 1, wherein the squamous cell lung cancer is stage IV.

3. The method of claim 1, wherein the squamous cell lung cancer is metastatic.

4. The method of claim 3, wherein the individual has distant metastases.

5. The method of claim 3, wherein the individual has disseminated metastases.

6. The method of claim 1, wherein the individual has malignant pleural or pericardial effusion.

7. The method of claim 1, wherein the individual has not received prior chemotherapy comprising 4-iodo-3-nitrobenzamide, a metabolite thereof, or a pharmaceutically acceptable salt thereof.

8. The method of claim 1, wherein the individual has not received prior chemotherapy comprising gemcitabine.

9. The method of claim 1, wherein the individual has not received prior chemotherapy comprising carboplatin.

10. The method of claim 1, wherein the individual has received prior chemotherapy comprising carboplatin in the adjuvant setting.

11. The method of claim 1, wherein the individual has squamous cell bronchogenic carcinoma.

12. The method of claim 2, wherein the individual has newly diagnosed stage IV squamous cell lung cancer.

13. The method of claim 2, wherein the individual has stage IV squamous cell lung cancer at diagnosis or recurrent after resection of the squamous cell lung cancer.

14. The method of claim 13, wherein the individual has previously received adjuvant chemotherapy for early stage lung cancer.

15. The method of claim 13, wherein the individual has not previously received adjuvant chemotherapy for early stage lung cancer.

16. The method of claim 2, wherein the individual has not previously received systemic treatment for advanced squamous cell lung cancer.

17. The method of claim 2, wherein the individual has previously received adjuvant therapy for early stage lung cancer and the adjuvant therapy ended at least 12 months ago.

18. The method of claim 2, wherein the individual has not previously received a therapy comprising chemotherapy and radiation therapy for locally advanced squamous cell lung cancer.

19. The method of claim 1, wherein the effective amount is administered over a 21-day treatment cycle, wherein (i) carboplatin is administered to the individual at 5 mg/ml·minute (AUC 5) on day 1 of the treatment cycle; (ii) gemcitabine is administered to the individual at 1000 mg/m² on days 1 and 8 of the treatment cycle; and (iii) 4-iodo-3-nitrobenzamide or a metabolite thereof or a pharmaceutically acceptable salt thereof is administered to the individual at 5.6 mg/kg on days 1, 4, 8, and 11 of the treatment cycle.

20. The method of claim 19, wherein the individual receives at least 2 treatment cycles.

21. The method of claim 1, wherein 4-iodo-3-nitrobenzamide or a metabolite thereof or a pharmaceutically acceptable salt thereof is administered intravenously to the individual.

22. The method of claim 1, wherein gemcitabine is administered intravenously to the individual.

23. The method of claim 1, wherein carboplatin is administered intravenously to the individual.

24. A kit comprising 4-iodo-3-nitrobenzamide, a metabolite thereof, or a pharmaceutically acceptable salt thereof, and instructions for using 4-iodo-3-nitrobenzamide, a metabolite thereof, or a pharmaceutically acceptable salt thereof in combination with gemcitabine and carboplatin for treatment of squamous cell lung cancer in an individual.

25. The kit of claim 24, wherein the instructions comprise administering an effective amount of (i) 4-iodo-3-nitrobenzamide, a metabolite thereof, or a pharmaceutically acceptable salt thereof, (ii) gemcitabine, and (iii) carboplatin over a 21-day treatment cycle to the individual, wherein (i) carboplatin is administered to the individual at 5 mg/ml·minute (AUC 5) on day 1 of the treatment cycle; (ii) gemcitabine is administered to the individual at 1000 mg/m² on days 1 and 8 of the treatment cycle; and (iii) 4-iodo-3-nitrobenzamide or a metabolite thereof or a pharmaceutically acceptable salt thereof is administered to the individual at 5.6 mg/kg on days 1, 4, 8, and 11 of the treatment cycle.

26. A kit comprising (i) 4-iodo-3-nitrobenzamide, a metabolite thereof, or a pharmaceutically acceptable salt thereof, (ii) gemcitabine, and (iii) carboplatin.

27. The kit of claim 26 further comprising instructions for using (i) 4-iodo-3-nitrobenzamide, a metabolite thereof, or a pharmaceutically acceptable salt thereof; (ii) gemcitabine; and (iii) carboplatin for treatment of squamous cell lung cancer in an individual.

28. The kit of claim 27, wherein the instructions comprise administering an effective amount of (i) 4-iodo-3-nitrobenzamide, a metabolite thereof, or a pharmaceutically acceptable salt thereof, (ii) gemcitabine, and (iii) carboplatin over a 21-day treatment cycle to the individual, wherein (i) carboplatin is administered to the individual at 5 mg/ml·minute (AUC 5) on day 1 of the treatment cycle; (ii) gemcitabine is administered to the individual at 1000 mg/m² on days 1 and 8 of the treatment cycle; and (iii) 4-iodo-3-nitrobenzamide or a metabolite thereof or a pharmaceutically acceptable salt thereof is administered to the individual at 5.6 mg/kg on days 1, 4, 8, and 11 of the treatment cycle.

29. The kit of claim 26, wherein the instructions are included in a package insert or a label in the kit.
